(19) United States
(12) Patent Application Publication
(10) Pub. No.: US 2020/0038450 A1
(43) Pub. Date: Feb. 6, 2020

(54) Tissue repair by stem cell recruitment and differentiation

(71) Applicant: The Trustees of Columbia University in the City of New York, New York, NY (US)

(72) Inventor: Chang Hun Lee, New York, NY (US)

(21) Appl. No.: 16/588,415

(22) Filed: Sep. 30, 2019

Related U.S. Application Data
(63) Continuation of application No. 15/558,015, filed on Sep. 13, 2017, now abandoned.
(60) Provisional application No. 62/303,915, filed on Mar. 4, 2016.
(60) Provisional application No. 62/133,171, filed on Mar. 13, 2015.

(51) Int. Cl. A61K 35/28; A61K 38/18; A61L 27/22; A61L 27/38; A61L 24/00; C12N 5/0775

(52) U.S. Cl. CPC A61K 35/28; A61K 38/1825; A61K 38/1841; A61L 27/225; A61L 2400/06; A61L 24/0031; C12N 5/0663; A61K 38/1875; A61K 38/18; A61L 27/3869

(57) ABSTRACT

Provided herein are compositions and methods for healing cartilage tissue defects or injury by forming fibrochondrocyte cells or fibrochondrocyte-like cells from recruited progenitor cells, such as mesenchymal stem cells.

### CROSS REFERENCE TO RELATED APPLICATIONS

The present application is a continuation of U.S. patent application Ser. No. 15/558,015, filed Sep. 13, 2017 which claims the benefit of priority to PCT International Application No. PCT/US16/22234 filed 13 Mar. 2016, which claims the benefit of U.S. Provisional Application Ser. No. 62/303,915 filed 4 Mar. 2016 and U.S. Provisional Application Ser. No. 62/133,171 filed 13 Mar. 2015; each of which are incorporated herein by reference in their entireties.

### STATEMENT REGARDING FEDERALLY SPONSORED RESEARCH OR DEVELOPMENT

Not Applicable.

### MATERIAL INCORPORATED-BY-REFERENCE

Not Applicable.

### FIELD OF THE INVENTION

The present disclosure generally relates to repair of cartilaginous tissue by temporal control of stem cell recruitment and differentiation.

### BACKGROUND OF THE INVENTION

The knee meniscus is an important fibrocartilaginous tissue that stabilizes and absorbs impact on the knee. The knee meniscus is semi-lunar and wedge-shaped fibrocartilaginous tissue between the distal femoral condyle and the proximal tibial plateaus in the knee joint. The meniscus plays a role in joint congruence, shock absorption, and stress transmission. The meniscus is characterized by its multiphase biochemical composition and structure. The outer third region (i.e., peripheral) of meniscus is vascularized and comprises a dense fibrous matrix populated with fibroblast-like cells. The inner third region comprises avascular cartilaginous tissue with chondrocyte-like cells. The middle region comprises intermediate fibrocartilaginous tissue, including a mixed population of fibroblast-like cells and chondrocyte-like cells. Despite recent attempts using matrix metalloproteinases (MMPs), biomaterials, and/or growth factors, no therapy exists to induce seamless healing of inner meniscus tears. As such, tears in the inner avascular region cannot be repaired by conventional methods and no regenerative therapy currently exists for avascular tears.

Clinically, over one million patients undergo surgical repair or meniscectomy each year in the United States. Meniscus injury or tearing can be more common in physically active populations including athletics and military service members. Tears in the vascularized outer third region of meniscus can be surgically repaired by suturing torn parts. In contrast, tears in the inner avascular region, similar to articular cartilage, cannot be repaired due to poor intrinsic healing capacity and the tears frequently extended into the middle-third region. Tears can often be accompanied by meniscus deterioration. Tears can frequently progress to meniscus deterioration. Poor healing of a tear occurring in the avascular region can deteriorate the meniscus. Partial or total meniscectomy is conventionally performed to alleviate symptoms caused by the irreparable meniscus injuries. However, meniscectomy significantly increases the incidence of osteoarthritis (OA) later in life by increasing joint contact stress. Meniscus injuries can be main contributing factors of osteoarthritis (OA). Approximately 50% of all patients with meniscal injuries develop OA within 10 to 20 years of injury.

Allograft transplantation from cadavers may be considered after meniscectomy to prevent the increase in joint contact pressure. But the use of allografts can have disadvantages, such as donor shortage, pathogen transmission, immunorejection, and tissue mis-match.

Various approaches have been tested to improve healing of avascular meniscus injuries. Fibrin clots and glues were used to simply bond torn meniscus pieces that somewhat improved healing in vitro and in vivo. Meniscal rasping, synovial flap implantation, and surgical induction of local blood supply have been applied to improve avascular meniscus healing. Combination dynamic loading and IL-1 enhanced integrative meniscal repair in explant culture model. More recently, juvenile meniscus fragments were implanted in injured sites and enhanced meniscus healing in vivo. Despite the findings, previous studies lacked biochemical and mechanical characterization of the healed tissues and produced controversial experimental outcomes. Seamless integration of cartilaginous matrix has not been achieved for inner meniscus tears.

Mesenchymal stem/progenitor cells (MSCs) from bone marrow or synovium have been the most popular cell types transplanted in vivo for meniscus healing. Allogeneic synovial MSCs infused in 1.5-mm cylindrical meniscus defects in the rabbit adhered to the injury site and apparently enhanced meniscal regeneration. Similarly, infusion of autologous MSCs into sheep meniscal defects engrafted and improved healing as evaluated by some, but far from comprehensive, parameters. Intra-articular injection of dual luciferase and LacZ tagged, synovium MSCs enhanced meniscus healing in the rat. However, several experimental studies have not shown an advantage to using stem cell transplantation in meniscus healing. Neither bone marrow nor platelet-rich plasma in hyaluronan-collagen scaffolds in surgically created circular rabbit meniscal punch defects showed any advantage in repair when compared to empty or acellular scaffolds.

### SUMMARY OF THE INVENTION

One aspect of the present disclosure provides method treating a subject having a tissue defect.

One embodiment provides a method treating a subject having a tissue defect. In some embodiments, the method includes providing a scaffold having a matrix material, a chemotactic factor or a profibrogenic factor having a first release duration; and a chondrogenic factor having a second release duration longer than the first release duration. Combining the factors with the scaffold or matrix can occur before or after delivery (e.g., dripping, coating, spraying, injecting, implanting) of the scaffold to the tissue defect. The method can also include delivering the scaffold to a tissue defect. The method can also include forming a fibrous matrix at the site of the tissue defect by a specified amount of time after scaffold delivery (e.g., by about 6-14 days, about 8-12 days, or about 10 days). The method can also include forming a fibrocartilaginous matrix comprising fibrochondrocyte cells or fibrochondrocyte-like cells integrated with tissue at the site of the tissue defect by a specified amount of time after scaffold delivery (e.g., by about by 4-10 weeks, about 5-8 weeks, or about 6 weeks).

Method features discussed above can be combined with other features discussed below.

In some embodiments, the scaffold having matrix, chemotactic factor or profibrogenic factor, and chondrogenic factor can be formed ex vivo. The method can include contacting the scaffold (containing matrix material) with the chemotactic factor or profibrogenic factor and the chondrogenic factor before delivering the scaffold to the tissue defect.

In some embodiments, the scaffold having matrix, chemotactic factor or profibrogenic factor, and chondrogenic factor can be formed in vivo. The method can include contacting the scaffold with the chemotactic factor or profibrogenic factor after delivering the scaffold to the tissue defect; and contacting the scaffold with the chondrogenic factor after delivering the scaffold to the tissue defect and during or after the release duration of the chemotactic factor or profibrogenic factor.

In some embodiments, the scaffold having matrix, chemotactic factor or profibrogenic factor, and chondrogenic factor can be formed ex vivo and in vivo. The method can include contacting the scaffold with the chemotactic factor or profibrogenic factor before delivering the scaffold to the tissue defect; and contacting the scaffold with the chondrogenic factor after delivering the scaffold to the tissue defect and during or after the release duration of the chemotactic factor or profibrogenic factor.

Features for forming the scaffold ex vivo or in vitro can be combined with other features discussed above and below.

In some embodiments, delivering the scaffold includes placing the scaffold in fluid communication with a progenitor cell (e.g., a precursor to a fibrochondrocyte or fibrochondrocyte-like cell, such as a mesenchymal stem cell or human mesenchymal stem cell) at the tissue defect site. Features related to fluid communication with a progenitor cell can be combined with other features discussed above and below.

In some embodiments, at least one chemotactic factor or a profibrogenic factor can be SDF-1, FGF-2, bFGF, CTGF, or GDF-5. In some embodiments, at least one chemotactic factor or a profibrogenic factor can be CTGF. In some embodiments, at least one chemotactic factor or profibrogenic factor is CTGF, and the CTGF is mixed with the matrix material (e.g., a fibrin glue or an injectable mussel-inspired biodegradable adhesive (iCMBA)). In some embodiments, the first release duration is a function of CTGF release rate from the matrix (e.g., fibrin glue or iCMBA). Features related to selection of chemotactic factor or a profibrogenic factor, matrix material, or delivery substrate can be combined with other features discussed above and below.

In some embodiments, at least one chondrogenic factor is TGF-β1, TGF-β3, IGF-1, and BMP-7. In some embodiments, at least one chondrogenic factor is TGF-β3. In some embodiments, at least one chondrogenic factor is TGF-β3, the TGF-β3 is encapsulated in polymeric microspheres, the encapsulated TGF-β3 is mixed with the matrix material (e.g., fibrin glue or iCMBA). In some embodiments, the second release duration is a function of TGF-β3 release rate from the polymeric microspheres. In some embodiments, the second release duration is a function of TGF-β3 release rate from the polymeric microspheres and the matrix (e.g., fibrin glue or iCMBA). Features related to selection of chondrogenic factor, matrix material, or delivery substrate can be combined with other features discussed above and below.

In some embodiments, the matrix material can include one or more of fibrin, fibrinogen, collagen, fibronectin, or iCMBA. Features related to selection of matrix material can be combined with other features discussed above and below.

In some embodiments, the first release duration of the chemotactic factor or profibrogenic factor is at least about 4 days up to about 10 days (e.g., about 5-8 days, or about 6 days). Features related to first release duration can be combined with other features discussed above and below.

In some embodiments, the second release duration of the chondrogenic factor is at least about 4 weeks (e.g., at least about 5 weeks, at least about 6 weeks). Features related to second release duration can be combined with other features discussed above and below.

In some embodiments, the scaffold can include an endogenous or exogenous cell introduced to the scaffold ex vivo or in vivo. In some embodiments, the scaffold includes an endogenous progenitor cell. In some embodiments, the scaffold does not include an exogenous progenitor cell. In some embodiments, the scaffold includes progenitor cell prior to scaffold delivery to the tissue defect site. In some embodiments, the scaffold does not include a progenitor cell until after scaffold delivery to the tissue defect site. In some embodiments, the scaffold includes an endogenous progenitor cell introduced to the scaffold in vivo or ex vivo. In some embodiments, the scaffold includes an exogenous progenitor cell introduced to the scaffold in vivo or ex vivo. Features related to cells in the scaffold can be combined with other features discussed above and below.

In some embodiments, the tissue defect site is at least partially located in an inner or avascular region of a cartilaginous tissue. In some embodiments, the tissue defect includes a tear, injury, osteoarthritis, or degeneration. In some embodiments, the tissue defect includes a longitudinal or vertical tear, a radial tear, a horizontal tear, a bucket handle tear, a parrot beak tear, or a flap tear. In some embodiments, the tissue (in which the defect is present) can be cartilaginous tissue, cartilage, a meniscus, a knee meniscus, a ligament, a ligament enthesis, a tendon, a tendon enthesis, an intervertebral disc, a temporomandibular joint (TMJ), a TMJ ligament, or a triangular fibrocartilage. Features related to tissue and tissue defect can be combined with other features discussed above and below.

In some embodiments, the chemotactic factor or the profibrogenic factor is applied at a concentration of about 1 to about 1000 ng/mL. For example, CTGF can be present at a concentration of about 100 ng/mL. In some embodiments, the chemotactic factor or the profibrogenic factor can be at a concentration of about 1 to about 100 μg/g of scaffold or matrix material. Features related to concentration of chondrogenic factor can be combined with other features discussed above and below.

In some embodiments, the chondrogenic factor is applied at a concentration of about 1 to about 1000 ng/mL. For example, TGFβ3 can be present at a concentration of about 10 ng/mL. In some embodiments, the chemotactic factor or the profibrogenic factor can be at a concentration of about 1 to about 100 μg/g of scaffold or matrix material. Features related to concentration of chondrogenic factor can be combined with other features discussed above and below.

Another aspect provides fibrocartilage tissue construct. In some embodiments, the fibrocartilage tissue construct includes a progenitor cell; an effective amount of a chemotactic factor or profibrogenic factor having a first release duration; an effective amount of a chondrogenic factor having a second release duration; and a scaffold comprising a matrix material; wherein, the first release duration is shorter than the second release duration; the effective amount of the chemotactic factor or the profibrogenic factor induces migration of a progenitor cell into or onto the scaffold when the scaffold is in fluid communication with the progenitor cell; and the effective amount of the chondrogenic factor in combination with the chemotactic factor or the profibrogenic factor induces formation of a fibrochondrocyte cell or a fibrochondrocyte-like cell from the progenitor cell. Features related to the fibrocartilage tissue construct can be combined with other features discussed above with respect to above methods of treating and the below method of manufacture.

Another aspect provides a method of forming a fibrocartilage tissue construct. In some embodiments, the method includes providing a scaffold comprising a matrix material; contacting the scaffold with a chemotactic factor or a profibrogenic factor; contacting the scaffold comprising the chemotactic factor or the profibrogenic factor with a chondrogenic factor; placing the scaffold in fluid communication with a progenitor cell; and delivering the scaffold to a tissue defect site; wherein the combination of the chemotactic factor or profibrogenic factor and the chondrogenic factor induces formation of a fibrochondrocyte cell or a fibrochondrocyte-like cell at the tissue defect site. Features related to the method of forming a fibrocartilage tissue construct can be combined with other features discussed above with respect to above methods of treating and the composition of a fibrocartilage tissue construct.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-FIG. 1D is a series of micrograph images showing a step-wise fibrochondrogenic differentiation of human bone marrow or synovium mesenchymal stem/progenitor cells (MSCs).

FIG. 1A shows MSCs formed a fibrous matrix with predominant COL-I expression after two weeks of CTGF treatment stained with Picrosirius Red.

FIG. 1B MSCs formed a fibrous matrix with predominant COL-I expression after two weeks of CTGF treatment stained with DAPI.

FIG. 1C shows fibrocartilaginous matrix stained with Alcian blue.

FIG. 1D shows proCOL-I⁺/proCOL-Iiα⁺ fibrochondrocyte-like cells. After two weeks treatment with connective tissue growth factor (CTGF), a profibrogenic cue, followed by 2 weeks transforming growth factor beta 3 (TGFβ3), a chondrogenic factor, can differentiate MSCs into proCOL-I⁺/proCOL-Iiα⁺ fibrochondrocyte-like cells (see e.g., FIG. 1D) that constitutes fibrocartilaginous matrix (see e.g., FIG. 1C).

FIG. 1E-FIG. 1H is a series of micrograph images showing MSCs treated with CTGF for 1 week and then TGFβ3 for 3 weeks. When MSCs were treated with CTGF treatment for 1 week and then TGFβ3 for 3 weeks (see e.g., FIG. 1E-FIG. 1H), COL-I expression increased by 1 week (see e.g., FIG. 1F) along with formation of intermediate fibrous matrix (see e.g., FIG. 1E), which transformed into hyaline-like cartilaginous matrix (see e.g., FIG. 1G), populated by pro-COL-Iiα⁺ and rounded chondrocyte-like cells (see e.g., FIG. 1H). PR: Picrosirius red; AB: Alcian blue; scale: 100 μm.

FIG. 1E shows the formation of intermediate fibrous matrix stained with Picrosirius red.

FIG. 1F shows COL-I expression stained with DAPI.

FIG. 1G shows hyaline-like cartilaginous matrix stained with Alcian blue.

FIG. 1H shows pro-COL-Iiα⁺ and rounded chondrocyte-like cells stained with DAPI.

FIG. 2A is an illustration depicting the strategy for inducing seamless healing of inter meniscus tears. Chemotaxis and/or profibrogenic factors (e.g., CTGF, FGF2, SDF-1, or GDF-5) are applied to the torn meniscus via fibrin glue to recruit synovium MSCs into the torn site, followed by a formation of fibrous integration. Then the intermediate fibrous matrix will be remodeled to cartilaginous tissue by inducing chondrogenic differentiation with growth factor treatment (e.g., TGF-β1, TGF-β3, IGF-1, GDF-5, or BMP-7).

FIG. 2B is an illustration depicting the strategy for inducing seamless healing of inter meniscus tears by regulating step-wise recruitment and differentiation of syMSCs.

FIG. 3A-FIG. 3B is a series of micrograph images showing tears in the inner third zone of a bovine meniscus explant model. Upon creation of the incision in the inner third zone by surgical blade and glued with fibrin gel, the meniscus explants were cultured in vitro. H&E (see e.g., FIG. 3A) and Safranin-O (Saf-O) (see e.g., FIG. 3B) staining of tissue sections showed no sign of healing or tissue integration in the inner meniscus tears for up to 6 weeks. Scale=200 μm.

FIG. 3A is a micrograph image of an H&E stained tissue section incised in the inner third zone and glued with fibrin gel after culturing for up to 6 weeks.

FIG. 3B is a micrograph image of a Saf-O stained tissue section incised in the inner third zone and glued with fibrin gel after culturing for up to 6 weeks.

FIG. 4A-FIG. 4C are a series of micrograph images at 10 days cultured on top of synovial MSCs showing synovial MSC recruitment into incised inner meniscus, followed by intermediate fibrous integration.

FIG. 4A is a micrograph image showing, upon CTGF (100 ng/mL) delivery through fibrin glue, synovial MSCs were recruited into the incised site after 10 days, identified by labeling human nucleus antigen (HNA). Scale=200 μm.

FIG. 4B is a micrograph images of an H&E stained section showing, at 10 days, incised meniscal tissues were integrated via fibrous matrix. Scale=200 μm.

FIG. 4C is a micrograph images of an Masson's Trichrome stained section showing, at 10 days, incised meniscal tissues were integrated via fibrous matrix. Scale=200 μm.

FIG. 4D-FIG. 4F are a series of micrograph images with fibrin alone without CTGF, no cell recruitment or tissue integration was observed. Scale=200 μm.

FIG. 4D is a micrograph image of a DAPI/human nucleus antigen (HNA) stained section showing no cell recruitment or tissue integration was observed with fibrin alone (i.e., no CTGF).

FIG. 4E is a micrograph image of an H&E stained section showing no cell recruitment or tissue integration was observed with fibrin alone (i.e., no CTGF).

FIG. 4F is a micrograph image of Masson's Trichrome stained section showing no cell recruitment or tissue integration was observed with fibrin alone (i.e., no CTGF).

FIG. 5A-FIG. 5F are a series of micrograph images showing results of fibrocartilaginous healing response of inner meniscus by sequential application of CTGF and TGFβ3 with and without fibrin with 6 weeks of TGFβ3 treatment. CTGF (100 ng/mL) was delivered via fibrin glue (100% released in 5 days; data not shown) and TGFβ3 (10 ng/mL) was exogenously applied from day 10.

FIG. 5A is a micrograph image of an H&E stained inner meniscus with fibrin alone, followed by TGFβ3 for 6 weeks failed to yield tissue integration.

FIG. 5B is a micrograph image of an H&E stained inner meniscus with a fibrin/CTGF-induced intermediate fibrous matrix integrating the incised tissues from recruited syMSCs showing successful remodeling into fibrocartilaginous matrix by TGFβ3 treatment. The fully integrated fibrocartilaginous matrix was populated by chondrocyte-like cells, reminiscent of native inner meniscus (see e.g., FIG. 5C).

FIG. 5C is a micrograph image of H&E stained native meniscus.

FIG. 5D is a micrograph image of a Safranin 0 stained inner meniscus healing with fibrin alone, followed by TGFβ3 for 6 weeks, failing to yield tissue integration.

FIG. 5E is a micrograph image of an Safranin 0 stained inner meniscus with a fibrin/CTGF-induced intermediate fibrous matrix integrating the incised tissues from recruited syMSCs was successfully remodeled into fibrocartilaginous matrix by TGFβ3 treatment. The fully integrated fibrocartilaginous matrix was populated by chondrocyte-like cells, reminiscent of native inner meniscus (see e.g., FIG. 5F).

FIG. 5F is a micrograph image of Safranin 0 stained native meniscus.

FIG. 6A-FIG. 6C is a series of micrograph images.

FIG. 6A is a micrograph image of the remaining defect in fibrin alone group.

FIG. 6B is a micrograph image of native-like structure and orientation of collagen fibrils in healed meniscus stained with Picrosirius Red at 6 weeks.

FIG. 6C is a micrograph image of native tissue stained with Picrosirius Red.

FIG. 7A-FIG. 7B is a series of images showing syMSC recruited into meniscus defect site in a bovine explant model.

FIG. 7A is a bright field image demonstrating that migrating synovium MSCs (arrows) toward the defect site of meniscus explants with CTGF/fibrin at day 1. 100 ng/mL CTGF was applied at the defect site via fibrin gel.

FIG. 7B is a bright field image of matrix filling at day 7 of a meniscus explant. 100 ng/mL CTGF was applied at the defect site via fibrin gel.

FIG. 8A-FIG. 8C is a series of images depicting the knee meniscus.

FIG. 8A is an illustration depicting the knee meniscus.

FIG. 8B is an illustration depicting the regionally variant cell/matrix phenotypes of the knee meniscus.

FIG. 8C is an illustration depicting the vascularity of the knee meniscus.

FIG. 9A-FIG. 9D is a series of images depicting meniscus regeneration by 3D printed scaffolds (Lee et al., Science Translational Medicine, 2014).

FIG. 9A is a micrograph image of regenerated knee meniscus.

FIG. 9B is a computer generated image of a knee meniscus scaffold.

FIG. 9C is an image of a knee meniscus scaffold.

FIG. 9D is an image of a knee meniscus scaffold.

FIG. 10 is a series of images showing examples of avascular meniscus tears (i.e., longitudinal or vertical tear, radial tear, horizontal tear, bucket handle tear, parrot beak tear, flap tear).

FIG. 11A is an illustration depicting the single application of CTGF-loaded fibrin glue mixed with PLGA microspheres (μS) encapsulating TGFβ3 process. The inner ⅓ portion of the bovine meniscus was cut, followed by a longitudinal incision in the middle of the meniscus explants. CTGF (100 mg/mL)-loaded fibrin glue with TGFβ3-μS (10 mg/mL) was then applied in the incised site and cultured for 6 weeks.

FIG. 11B is a scatter plot depicting the in vitro release profile showing fast release of CTGF within 5 days and sustained release of TGFβ3 from PLGA μS.

FIG. 12 is a series of microscopy images showing meniscus healing at 6 weeks by syMSC recruitment. FIG. 12 shows avascular meniscus healing by CTGF-loaded fibrin glue mixed with PLGA μS-encapsulating TGFβ3. CTGF-loaded fibrin glue with TGFβ3-μS successfully led to seamless healing of avascular meniscus incision. However, fibrin alone, fibrin with CTGF, and fibrin with TGFβ3-μS failed to lead to integration of the torn meniscus tissues. Scale=200 μm.

FIG. 13 is a series of scatter plots showing meniscus healing at 6 weeks by syMSC recruitment.

FIG. 14 is an image and a series of bar graphs showing nanoindentation: modulus mapping.

FIG. 15 is a depiction of the advanced Bio-glue.

FIG. 16 is an image of a meniscus using injectable mussel-inspired biodegradable adhesives (iCMBA)+CTGF+TGFβ3.

FIG. 17 is an image of in vivo meniscus in a rabbit and a dog.

### DETAILED DESCRIPTION OF THE INVENTION

The present disclosure is based, at least in part, on the discovery that temporal control of stem cell recruitment and step-wise fibrocartilaginous differentiation leads to seamless healing of inner meniscus tears.

A novel and efficient strategy, as described herein, induces seamless healing of the avascular meniscus in three sequential stages. First, endogenous stem/progenitor cells (e.g., mesenchymal stem cells (MSCs)) are recruited by delivering profibrogenic factors such as connective tissue growth factor (CTGF) via fibrin glue to the site of injury. Second, the combination of fibrin and CTGF induces the formation of an integrated intermediate fibrous matrix at the site of injury. Third, cartilaginous remodeling into the zone-specific fibrocartilage was developed by exogenously applying chondrogenic factors such as transforming growth factor beta 3 (TGFβ3) (e.g., for 6 weeks) to differentiate the intermediate fibrous matrix into fibrocartilaginous matrix.

More specifically, it was discovered that mesenchymal stem/progenitor cells (MSCs) undergo a step-wise fibrochondrogenic differentiation: intermediate fibrogenic differentiation, followed by chondrogenic differentiation. As described herein, the novel approach achieves seamless healing of inner meniscus tears by endogenous stem/progenitor cells. The innovative strategy guides integrated tissue remodeling in cartilaginous zone of meniscus by temporally controlled stem cell recruitment, intermediate fibrogenesis, and fibrocartilaginous remodeling. As described herein, the disclosed subject matter can be an effective approach to inner meniscus healing, consequently preventing the early onset of osteoarthritis.

The disclosed strategy improved meniscal tissue healing and demonstrated native-like cells populating the site of injury in vitro. Thus, this approach can provide an efficient and efficacious approach for healing injuries in the avascular meniscus, preventing the need for meniscectomies.

In some embodiments, and in contrast to conventional methods, the present disclosure provides, solutions to several problems such as (1) whether transplanted stem cells differentiated into fibrochondrocytes; (2) if healed meniscus-like tissues had zone-specific fibrocartilaginous matrix distribution; and (3) key factors that promote stem/progenitor cells to differentiate along a fibrochondrocyte lineage in vitro and in vivo.

In some embodiments, and in contrast to conventional methods, the present disclosure achieved seamless healing of avascular meniscus tears by the sequential treatment of growth factors, and guided step-wise recruitment and differentiation of stem cells. The disclosed strategy leads to a significant improvement in avascular meniscus healing, avoiding problems faced by cell isolation or transplantation to achieve.

In some embodiments, and in contrast to conventional methods (i.e., using cell transplantation, for healing or for regeneration by endogenous cell recruitment), the disclosed strategy can serve as a direct, readily translational approach by overcoming the limitations related to invasive cell isolation, ex vivo cell culture, packaging, shipment, or regulatory barriers, for example.

Compositions or methods described herein can be adapted to, used in combination with, used concurrently with, used in parallel, used sequentially, or used as a replacement of one or more compositions or steps of the disclosure of U.S. patent application Ser. No. 13/877,260, published as U.S. Pat Pub No. 2014-0079739, incorporated herein by reference.

Progenitor Cells

A progenitor cell, as that term is used herein, is a precursor to a fibrochondrocyte or fibrochondrocyte-like cell and can differentiate in the presence of CTGF and TGFβ3. A progenitor cell can be a multipotent cell. A progenitor cell can be self-renewing. For example, a progenitor cell can be a mesenchymal stem cell (e.g., a human mesenchymal stem cell). The progenitor cell can be substantially less differentiated than a fibrochondrocyte or fibrochondrocyte-like cell.

As described herein, a progenitor cell can be an endogenous progenitor cell or an exogenous progenitor cell. For example, a progenitor cell can be freshly isolated or not pre-treated with growth factors before being further cultured with compositions including CTGF or TGFβ3 described herein. Progenitor cells can be isolated, purified, or cultured by a variety of means known to the art. Methods for the isolation and culture of tissue progenitor cells are discussed in, for example, Vunjak-Novakovic and Freshney (2006) Culture of Cells for Tissue Engineering, Wiley-Liss, ISBN 0471629359. For example, mesenchymal stem cells can be isolated from bone marrow and culture-expanded as described in U.S. patent application Ser. No. 13/877,260, published as U.S. Pat Pub No. 2014-0079739, incorporated herein by reference.

In various embodiments, a progenitor cell is a precursor to a fibrochondrocyte or fibrochondrocyte-like cell and differentiates under conditions including sequential or concurrent provision of CTGF or TGFβ3, as described herein. In some embodiments, a progenitor cell does not display a fibrocartilaginous matrix. For example, a progenitor cell may not display COL deposition. In some embodiments, a progenitor cell does not display a fibrochondrocyte-specific marker, such as proCOL-I+ or proCOL-IIα+.

In some embodiments, the tissue progenitor cells can be derived from the same or different species as the transplant recipient. For example, the progenitor cells can be derived from an animal, including, but not limited to, mammal, reptile, or avian, more preferably horses, cows, dogs, cats, sheep, pigs, or chickens, or most preferably human.

Progenitor cells can be cultured by a variety of means known to the art or as described in U.S. patent application Ser. No. 13/877,260, published as U.S. Pat Pub No. 2014-0079739, and incorporated herein by reference. For example, progenitor cells can be plated (e.g., about 100,000 cells per well) for 2D culture. As another example, progenitor cells can be centrifuged (e.g., about 2 million cells) to form a 3D pellet. Monolayer (2D) or 3D cell pellets can be cultured in a growth medium. Monolayer (2D) or 3D cell pellets can be treated with CTGF or TGFβ3 sequentially.

Fibrochondrocyte or Fibrochondrocyte-Like Cells

In various embodiments, a fibrochondrocyte or fibrochondrocyte-like cell differentiates from a progenitor cell under conditions including sequential provision of CTGF or TGFβ3 described herein. In some embodiments, a fibrochondrocyte or fibrochondrocyte-like cell displays a fibrocartilaginous matrix. For example, a fibrochondrocyte or fibrochondrocyte-like cell can display COL deposition. In some embodiments, a fibrochondrocyte or fibrochondrocyte-like cell can display a fibrochondrocyte-specific marker, such as proCOL-I+ or proCOL-IIα+.

Differentiation Methods

Described herein are exemplary methods to induce fibrochondrogenic differentiation of progenitor cells by sequential or concurrent treatment of growth factors, such as connective tissue growth factor (CTGF) and transforming growth factor β3 (TGFβ3) on substrates (i.e., matrix materials) as described herein and in U.S. patent application Ser. No. 13/877,260, published as U.S. Pat Pub No. 2014-0079739, incorporated herein by reference.

Chemotactic or Profibrogenic Factors.

As shown herein, transient delivery of chemotactic factor or a profibrogenic factor and sustained delivery of a chondrogenic factor successfully recruited synovial MSCs into tissue defect sites and led to integrated healing with fibrocartilaginous tissue. A chemotactic or profibrogenic factor can include SDF-1, FGF-2, bFGF, CTGF, or GDF-5. For example, a chemotactic or profibrogenic factor can be CTGF.

CTGF is available from a variety of commercial sources (e.g., Santa Cruz Biotechnology, Inc., Santa Cruz, Calif.). The connective tissue growth factor can be human connective tissue growth factor (e.g., Accession No. NP_001892).

The chemotactic or profibrogenic factor can be supplied at, for example, a concentration of about 0 to about 1000 ng/mL. For example, the chemotactic or profibrogenic factor (e.g., CTGF) can be present at a concentration of about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL, about 180 ng/mL, about 190 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, about 700 ng/mL, about 750 ng/mL, about 800 ng/mL, about 850 ng/mL, about 900 ng/mL, about 950 ng/mL, or about 1000 ng/mL. It is understood that recitation of the above discrete values includes a range between each recited value. For example, CTGF can be present at a concentration of about 100 ng/mL (see e.g., Example 1, Example 4).

The chemotactic or profibrogenic factor can be supplied by a delivery substrate at, for example, a concentration of about 0 to about 2500 μg per 250 mg substrate or 0 to 100 μg/g. For example, a chemotactic or profibrogenic factor (e.g., CTGF) can be present in a delivery substrate at a concentration of about 1 μg/g, about 2 μg/g, about 3 μg/g, about 4 μg/g, about 5 μg/g, about 6 μg/g, about 7 μg/g, about 8 μg/g, about 9 μg/g, about 10 μg/g, about 11 μg/g, about 12 μg/g, about 13 μg/g, about 14 μg/g, about 15 μg/g, about 16 μg/g, about 17 μg/g, about 18 μg/g, about 19 μg/g, about 20 μg/g, about 21 μg/g, about 22 μg/g, about 23 μg/g, about 24 μg/g, about 25 μg/g, about 26 μg/g, about 27 μg/g, about 28 μg/g, about 29 μg/g about 30 μg/g, about 31 μg/g, about 32 μg/g, about 33 μg/g, about 34 μg/g, about 35 μg/g, about 36 μg/g, about 37 μg/g, about 38 μg/g, about 39 μg/g about 40 μg/g, about 41 μg/g, about 42 μg/g, about 43 μg/g, about 44 μg/g, about 45 μg/g, about 46 μg/g, about 47 μg/g, about 48 μg/g, about 49 μg/g about 50 μg/g, about 51 μg/g, about 52 μg/g, about 53 μg/g, about 54 μg/g, about 55 μg/g, about 56 μg/g, about 57 μg/g, about 58 μg/g, about 59 μg/g about 60 μg/g, about 61 μg/g, about 62 μg/g, about 63 μg/g, about 64 μg/g, about 65 μg/g, about 66 μg/g, about 67 μg/g, about 68 μg/g, about 69 μg/g about 70 μg/g, about 71 μg/g, about 72 μg/g, about 73 μg/g, about 74 μg/g, about 75 μg/g, about 76 μg/g, about 77 μg/g, about 78 μg/g, about 79 μg/g about 80 μg/g, about 81 μg/g, about 82 μg/g, about 83 μg/g, about 84 μg/g, about 85 μg/g, about 86 μg/g, about 87 μg/g, about 88 μg/g, about 89 μg/g about 90 μg/g, about 91 μg/g, about 92 μg/g, about 93 μg/g, about 94 μg/g, about 95 μg/g, about 96 μg/g, about 97 μg/g, about 98 μg/g, about 99 μg/g or about 100 μg/g. It is understood that recitation of the above discrete values includes a range between each recited value.

A chemotactic or profibrogenic factor can be supplied for a specific period of time. For example, a chemotactic or profibrogenic factor (e.g., CTGF) can be released from a delivery substrate over the course of about 3 days to about 14 days. For example, delivery duration of chemotactic or profibrogenic factor (e.g., CTGF) can be from about 4 days to about 10 days (see Example 4). As another example, CTGF administration can occur over about 3 days to about 14 days.

For example, a chemotactic or profibrogenic factor can be supplied for less than or about 1 day, less than or about 2 days, less than or about 3 days, less than or about 4 days, less than or about 5 days, less than or about 6 days, less than or about 7 days, less than or about 8 days, less than or about 9 days, or less than or about 10 days. It is understood that recitation of the above discrete values includes a range between each recited value. For example, a chemotactic or profibrogenic factor (e.g., CTGF) can be administered by a release delivery substrate (e.g., comprising fibrin glue) that releases a majority of or substantially all of the chemotactic or profibrogenic factor within about 6 days (see e.g., Example 4, Example 5).

Administration of the chemotactic or profibrogenic factor (e.g., CTGF) can be a single event or a plurality of sequential events. It is understood that recitation of the above discrete values includes a range between each recited value.

As described herein, integration of a fibrous matrix at the site of injury after delivery of the chemotactic or profibrogenic factor can be observed at least about 1 day to at least about 10 days. For example, integration of a fibrous matrix at the site of injury after delivery of the chemotactic or profibrogenic factor can be observed at least about 1 day, at least about 2 days, at least about 3 days, at least about 4 days, at least about 5 days, at least about 6 days, at least about 7 days, at least about 8 days, at least about 9 days, or at least about 10 days, or more. It is understood that recitation of the above discrete values includes a range between each recited value.

Chondrogenic Factors.

As shown herein, transient delivery of chemotactic factor or a profibrogenic factor and sustained delivery of a chondrogenic factor successfully recruited synovial MSCs into tissue defect sites and led to integrated healing with fibrocartilaginous tissue.

A chondrogenic factor can include TGF-β (e.g., TGF-β1 or TGF-β3), IGF-1, or BMP-7. For example, a chondrogenic factor can be TGF-β3.

TGFβ3 is available from a variety of commercial sources (e.g., Santa Cruz Biotechnology, Inc., Santa Cruz, Calif.). The connective tissue growth factor can be human TGFβ3 (e.g., Accession No. NM_003239) (e.g., Avotermin).

The chondrogenic factor can be supplied at, for example, a concentration of about 0 to about 1000 ng/mL. For example, the chondrogenic factor (e.g., TGFβ3) can be present at a concentration of about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL, about 180 ng/mL, about 190 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, or about 1000 ng/mL. It is understood that recitation of the above discrete values includes a range between each recited value. For example, TGFβ3 can be present at a concentration of about 10 ng/mL (see e.g., Example 1, Example 4).

The chondrogenic factor can be supplied by a delivery substrate at, for example, a concentration of about 0 to about 2500 μg per 250 mg substrate or 0 to 100 μg/g. For example, a chondrogenic factor (e.g., TGFβ3) can be present in a delivery substrate at a concentration of about 1 μg/g, about 2 μg/g, about 3 μg/g, about 4 μg/g, about 5 μg/g, about 6 μg/g, about 7 μg/g, about 8 μg/g, about 9 μg/g, about 10 μg/g, about 11 μg/g, about 12 μg/g, about 13 μg/g, about 14 μg/g, about 15 μg/g, about 16 μg/g, about 17 μg/g, about 18 μg/g, about 19 μg/g, about 20 μg/g, about 21 μg/g, about 22 μg/g, about 23 μg/g, about 24 μg/g, about 25 μg/g, about 26 μg/g, about 27 μg/g, about 28 μg/g, about 29 μg/g about 30 μg/g, about 31 μg/g, about 32 μg/g, about 33 μg/g, about 34 μg/g, about 35 μg/g, about 36 μg/g, about 37 μg/g, about 38 μg/g, about 39 μg/g about 40 μg/g, about 41 μg/g, about 42 μg/g, about 43 μg/g, about 44 μg/g, about 45 μg/g, about 46 μg/g, about 47 μg/g, about 48 μg/g, about 49 μg/g about 50 μg/g, about 51 μg/g, about 52 μg/g, about 53 μg/g, about 54 μg/g, about 55 μg/g, about 56 μg/g, about 57 μg/g, about 58 μg/g, about 59 μg/g about 60 μg/g, about 61 μg/g, about 62 μg/g, about 63 μg/g, about 64 μg/g, about 65 μg/g, about 66 μg/g, about 67 μg/g, about 68 μg/g, about 69 μg/g about 70 μg/g, about 71 μg/g, about 72 μg/g, about 73 μg/g, about 74 μg/g, about 75 μg/g, about 76 μg/g, about 77 μg/g, about 78 μg/g, about 79 μg/g about 80 μg/g, about 81 μg/g, about 82 μg/g, about 83 μg/g, about 84 μg/g, about 85 μg/g, about 86 μg/g, about 87 μg/g, about 88 μg/g, about 89 μg/g about 90 μg/g, about 91 μg/g, about 92 μg/g, about 93 μg/g, about 94 μg/g, about 95 μg/g, about 96 μg/g, about 97 μg/g, about 98 μg/g, about 99 μg/g or about 100 μg/g. It is understood that recitation of the above discrete values includes a range between each recited value. For example, TGFβ3 can be present at a concentration of about 10 μg/g (see e.g., Example 5).

A chondrogenic factor can be supplied for a specific period of time. For example, a chondrogenic factor (e.g., TGFβ3) can be released from a delivery substrate over the course of about 1 week to about 10 weeks, or more. For example, delivery duration of a chondrogenic factor (e.g., TGFβ3) can occur over about one week, about two weeks, about three weeks, about four weeks, about five weeks, about six weeks, about seven weeks, about eight weeks, about nine weeks, or about 10 weeks, or more. As another example, TGFβ3 administration can occur over about one week to six weeks.

In some embodiments, the duration of sustained delivery of a chondrogenic factor (e.g., TGFβ3) can be about 5 days or more. For example, the delivery duration of a chondrogenic factor (e.g., TGFβ3) can be about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 50 days, or more. It is understood that recitation of the above discrete values includes a range between each recited value.

As described herein, successfully remodeling into native-like fibrocartilaginous tissue at the site of injury after sustained delivery of the chondrogenic factor (e.g., TGFβ3) can be observed by about 6 weeks (see e.g., Example 4, Example 5). For example, remodeling into native-like fibrocartilaginous tissue at the site of injury after delivery of chondrogenic factor (e.g., TGFβ3) can be can be observed by about 4 weeks, about 5 weeks, about 6 weeks, about 7 weeks, or about 8 weeks. It is understood that recitation of the above discrete values includes a range between each recited value.

Delivery Substrates.

Various delivery strategies can be applied, including local delivery, controlled release, or exogenous treatment, to find the optimal doses, sequence, or duration of each growth factor (e.g., chemotactic, profibrogenic, or chondrogenic factors) leading to the seamless healing of inner meniscus tears.

Exemplary experiments show that transient delivery of CTGF via fibrin gel successfully recruited synovial MSCs into defect site and formed intermediate fibrous integration within 10 days (see Example 4). Exemplary experiments further showed the initial fibrous matrix in healing region was then successfully remodeled into native-like fibrocartilaginous tissue upon 6 weeks TGFβ3 treatment. Further experiments over one embodiment showed that a short-term delivery of CTGF (e.g., for 1-4 days) was not sufficient to induce fibrous tissue integration, whereas a prolonged CTGF application (e.g., over 10 days) attenuated fibrocartilaginous remodeling after TGFβ3 treatment (see Example 4).

Further exemplary experiments showed transient delivery of CTGF (e.g., over the course of 6 days) via fibrin gel and sustained release of TGFβ3 from PLGA μS (e.g., over the course of 35 days) successfully recruited synovial MSCs into the defect sites and lead to integrated healing with fibrocartilaginous tissue by 6 weeks (see Example 5).

As evident by the exemplary experiments described above, some embodiments include a first delivery substrate and a second delivery substrate, where the first delivery substrate provides a relatively faster or transient delivery and the second delivery substrate provides a relatively slower or sustained delivery. In some embodiments, a release rate can be quantified as a number of days needed to release a majority of or substantially all of the factor from the delivery substrate.

A delivery substrate can comprise a matrix material (as defined herein) or be a separate material in or on a matrix material. For example, a delivery substrate can be a matrix material, such as fibrin glue. As another example, a delivery substrate can be an encapsulation material, such as polymeric microspheres. As another example, a first delivery substrate can be a matrix material, such as fibrin glue; and a second delivery substrate can be an encapsulation material, such as polymeric microspheres, where the encapsulation material is comprised by (e.g., mixed or combined with) the matrix material. As another example, a first delivery substrate can be a first encapsulation material and the second delivery substrate can be a second encapsulation material, where both delivery substrates are comprised by (e.g., mixed or combined with) the matrix material.

In some embodiments, the substrate (i.e., matrix material) can be a temporary substrate. For example, a temporary substrate can be hydrogel. The substrate can be suitable for cell migration, tissue formation, delivery of cells, or delivery of biochemical cues (e.g., chemotactic or profibrogenic factors). The substrate can be a fibrin gel or a fibrin glue. For example, the substrate can be a higher density fibrin gel or higher density fibrin glue. Higher density fibrin can provide sustained, long-term delivery of chondrogenic cues.

For example, the delivery substrate (i.e., matrix material) can be a controlled delivery vehicle. As described herein, the controlled delivery vehicle can be microspheres (μS). For example, the delivery vehicle can be a polymeric microsphere, e.g., a PLGA polymeric microspheres. A variety of polymeric delivery systems, as well as methods for encapsulating a molecule such as a growth factor, are known to the art (see e.g., Varde and Pack 2004 Expert Opin Biol Ther 4, 35-51). Polymeric microspheres can be produced using naturally occurring or synthetic polymers and are particulate systems in the size range of 0.1 μm to 500 μm. Polymeric micelles and polymeromes are polymeric delivery vehicles with similar characteristics to microspheres and can also facilitate encapsulation and matrix integration of the compounds described herein. Fabrication, encapsulation, and stabilization of microspheres for a variety of payloads are within the skill of the art (see e.g., Varde & Pack (2004) Expert Opin. Biol. 4(1) 35-51). The release rate of the microspheres can be tailored by type of polymer, polymer molecular weight, copolymer composition, excipients added to the microsphere formulation, and microsphere size. Polymer materials useful for forming microspheres include PLA, PLGA, PLGA coated with DPPC, DPPC, DSPC, EVAc, gelatin, albumin, chitosan, dextran, DL-PLG, SDLMs, PEG (e.g., ProMaxx), sodium hyaluronate, diketopiperazine derivatives (e.g., Technosphere), calcium phosphate-PEG particles, and/or oligosaccharide derivative DPPG (e.g., Solidose). Encapsulation can be accomplished, for example, using a water/oil single emulsion method, a water-oil-water double emulsion method, or lyophilization. Several commercial encapsulation technologies are available (e.g., ProLease®, Alkerme). Selection of an encapsulation agent can depend on the desired release rate.

For example, the substrate (i.e., matrix material) can be a higher density fibrin gel or higher density fibrin glue. Higher density fibrin can provide sustained, long-term delivery of chondrogenic cues.

A delivery substrate can provide (a) a relatively faster or transient delivery or (b) a relatively slower or sustained delivery.

In some embodiments, a first delivery substrate provides a relatively faster or transient delivery and a second delivery substrate provides a relatively slower or sustained delivery. For example, the fast release delivery substrate (i.e., matrix material) can be fibrin glue or gel. As another example, the fast release delivery substrate can be microspheres (μS).

The release rate can be less than about 10 days. In some embodiments, release rate can be quantified as a number of days needed to release a majority of or substantially all of the chemotactic or profibrogenic factor. For example, a fast release rate can be less than or about 1 day, less than or about 2 days, less than or about 3 days, less than or about 4 days, less than or about 5 days, less than or about 6 days, less than or about 7 days, less than or about 8 days, less than or about 9 days, or less than or about 10 days. It is understood that recitation of the above discrete values includes a range between each recited value. For example, a chemotactic or profibrogenic factor (e.g., CTGF) can be administered by a release delivery substrate (e.g., comprising fibrin glue) that releases a majority of or substantially all of the chemotactic or profibrogenic factor within about 6 days (see e.g., Example 4, Example 5).

In some embodiments, the delivery substrate (i.e., matrix material) can be a relatively slower release or sustained delivery substrate. For example, the slow release delivery substrate can be microspheres (μS). As another example, the slow release delivery substrate can be a matrix material. The slow release rate can be about 5 days or more. For example, the slow release rate can be about 5 days or more, about 6 days or more, about 7 days or more, about 8 days or more, about 9 days or more, about 10 days or more, about 11 days or more, about 12 days or more, about 13 days or more, about 14 days or more, about 15 days or more, about 16 days or more, about 17 days or more, about 18 days or more, about 19 days or more, about 20 days or more, about 21 days or more, about 22 days or more, about 23 days or more, about 24 days or more, about 25 days or more, about 26 days or more, about 27 days or more, about 28 days or more, about 29 days or more, about 30 days or more, about 31 days or more, about 32 days or more, about 33 days or more, about 34 days or more, about 35 days or more, about 36 days or more, about 37 days or more, about 38 days or more, about 39 days or more, about 40 days or more, about 41 days or more, about 42 days or more, about 43 days or more, about 44 days or more, about 45 days or more, about 46 days or more, about 47 days or more, about 48 days or more, about 49 days or more, about 50 days or more. It is understood that recitation of the above discrete values includes a range between each recited value and that each value can represent a discrete minimum or a discrete maximum. For example, a chondrogenic factor (e.g., TGFβ3) can be administered by a microencapsulated delivery vehicle (e.g., PLGA microspheres) that releases a majority of or substantially all of the chondrogenic factor within about 35 days (see e.g., Example 5).

As another example, the slow release rate can be more than about one week, more than about two weeks, more than about three weeks, more than about four weeks, more than about five weeks, or more than about six weeks, or more. It is understood that recitation of the above discrete values includes a range between each recited value.

For example, a slow release delivery substrate can be a higher density of fibrin gel, fibrin glue, or controlled delivery vehicle. As another example, controlled delivery vehicle can be microspheres (e.g., PLGA). As another example, other controlled delivery vehicles can be utilized if sustained, long-term delivery of chondrogenic cues is necessary.

Compositions including chemotactic or profibrogenic factors (e.g., CTGF) or chondrogenic factors (e.g., TGFβ3) can be formulated or encapsulated for controlled release, as described further below. For example, compositions including CTGF or TGFβ3 can be encapsulated in microspheres, such as PLGA microspheres as described in U.S. patent application Ser. No. 13/877,260, published as U.S. Pat Pub No. 2014-0079739, incorporated herein by reference. Differing PLGA ratios can be used to provide sequential release of CTGF or TGFβ3. For example, a composition including CTGF can be encapsulated in 50:50 PLGA microspheres. As another example, a composition including TGFβ3 can be encapsulated in 75:25 PLGA microspheres. Encapsulated compositions including CTGF or TGFβ3 can be embedded in a biocompatible matrix A (e.g., a 3D fibrin gel) loaded with progenitor cells (e.g., mesenchymal stem cells) and cultured in vitro.

As described herein, after chemotactic or profibrogenic factor treatment, induction of chondrogenic differentiation at the site of injury can be observed after treating with a chondrogenic factor for at least about 1 day to at least about 10 days. For example, after chemotactic or profibrogenic factor treatment, induction of chondrogenic differentiation at the site of injury can be observed after treating with a chondrogenic factor for at least about 1 day, at least about 2 days, at least about 3 days, at least about 4 days, at least about 5 days, at least about 6 days, at least about 7 days, at least about 8 days, at least about 9 days, or at least about 10 days, or more. It is understood that recitation of the above discrete values includes a range between each recited value.

As described herein, a fully integrated fibrocartilaginous matrix is observed at the site of injury after starting the sequential treatment of chemotactic or profibrogenic factor and a chondrogenic factor from at least about 1 week to at least about 6 weeks, or more. For example, a fully integrated fibrocartilaginous matrix is observed at the site of injury after starting the sequential treatment of chemotactic or profibrogenic factor and a chondrogenic factor from at least about 1 week, at least about 2 weeks, at least about 3 weeks, at least about 4 weeks, at least about 5 weeks, at least about 6 weeks, at least about 7 weeks, at least about 8 weeks, or more. It is understood that recitation of the above discrete values includes a range between each recited value.

As described herein, the sequential application of the multiple factors can be applied by combining various hydrogel-based carriers or biodegradable polymeric controlled delivery vehicles.

Scaffold

Various embodiments described herein employ a scaffold or matrix material (e.g., a substrate). For example, a composition including CTGF or TGFβ3 can be included in or on a scaffold. A scaffold or matrix material, as described herein, can be introduced at a defect site in a cartilaginous tissue. A defect site can include an injury, a tear, or deterioration. A defect site can be at least partially located in the inner or avascular region of a cartilaginous tissue.

The scaffold optionally does not comprise a transplanted mammalian cell, i.e., no cell is applied to the scaffold; any cell present in the scaffold migrated into the scaffold.

A scaffold can be fabricated with any matrix material recognized as useful by the skilled artisan. A matrix material can be a biocompatible material that generally forms a porous, microcellular scaffold, which provides a physical support for cells migrating thereto. Such matrix materials can: allow cell attachment or migration; deliver or retain cells or biochemical factors; enable diffusion of cell nutrients or expressed products; or exert certain mechanical or biological influences to modify the behavior of the cell phase. The matrix material generally forms a porous, microcellular scaffold of a biocompatible material that provides a physical support or an adhesive substrate for recruitment or growth of cells during in vitro or in vivo culturing.

Suitable scaffold or matrix materials are discussed in, for example, Ma and Elisseeff, ed. (2005) Scaffolding In Tissue Engineering, CRC, ISBN 1574445219; Saltzman (2004) Tissue Engineering: Engineering Principles for the Design of Replacement Organs and Tissues, Oxford ISBN 019514130X. For example, matrix materials can be, at least in part, solid xenogenic (e.g., hydroxyapatite) (Kuboki et al. 1995 Connect Tissue Res 32, 219-226; Murata et al. 1998 Int J Oral Maxillofac Surg 27, 391-396), solid alloplastic (polyethylene polymers) materials (Saito and Takaoka 2003 Biomaterials 24 2287-93; Isobe et al. 1999 J Oral Maxillofac Surg 57, 695-8), or gels of autogenous (Sweeney et al. 1995. J Neurosurg 83, 710-715), allogenic (Bax et al. 1999 Calcif Tissue Int 65, 83-89; Viljanen et al. 1997 Int J Oral Maxillofac Surg 26, 389-393), or alloplastic origin (Santos et al. 1998. J Biomed Mater Res 41, 87-94), or combinations of the above (Alpaslan et al. 1996 Br J of Oral Maxillofac Surg 34, 414-418).

The matrix comprising the scaffold can have an adequate porosity and an adequate pore size so as to facilitate cell recruitment and diffusion throughout the whole structure of both cells and nutrients. The matrix can be biodegradable providing for absorption of the matrix by the surrounding tissues, which can eliminate the necessity of a surgical removal. The rate at which degradation occurs can coincide as much as possible with the rate of tissue or organ formation. Thus, while cells are fabricating their own natural structure around themselves, the matrix can provide structural integrity and eventually break down, leaving the neotissue, newly formed tissue or organ which can assume the mechanical load. The matrix can be an injectable matrix in some configurations. The matrix can be delivered to a tissue using minimally invasive endoscopic procedures.

The scaffold can comprise a matrix material having different phases of viscosity. For example, a matrix can have a substantially liquid phase or a substantially gelled phase. The transition between phases can be stimulated by a variety of factors including, but limited to, light, chemical, magnetic, electrical, and mechanical stimulus. For example, the matrix can be a thermosensitive matrix with a substantially liquid phase at about room temperature and a substantially gelled phase at about body temperature. The liquid phase of the matrix can have a lower viscosity that provides for optimal distribution of growth factors or other additives and injectability, while the solid phase of the matrix can have an elevated viscosity that provides for matrix retention at or within the target tissue.

The scaffold can comprise one or more layers, each with the same or different matrix materials. For example, a scaffold can comprises at least two layers, at least three layers, at least four layers, or more. As another example, a scaffold can comprise a first layer comprising a first matrix material and a second layer comprising a second matrix material. As another example, a scaffold can comprise a first layer comprising a chemotactic factor or profibrogenic factor and a second layer comprising a chondrogenic factor. As another example, a scaffold can comprise a first layer comprising a chemotactic factor or profibrogenic factor and a chondrogenic factor and a second layer comprising a chondrogenic factor. As another example, a scaffold can comprise a first layer comprising a chemotactic factor or profibrogenic factor and a second layer comprising a chondrogenic factor and chemotactic factor or profibrogenic factor. As another example, a scaffold can comprise a first layer comprising CTGF and a second layer comprising TGFβ3. As another example, a scaffold can comprise a first layer comprising CTGF and TGFβ3 and a second layer comprising TGFβ3. As another example, a scaffold can comprise a first layer comprising CTGF and a second layer comprising TGFβ3 and CTGF.

The scaffold can comprise a matrix material formed of synthetic polymers. Such synthetic polymers include, but are not limited to, polyurethanes, polyorthoesters, polyvinyl alcohol, polyamides, polycarbonates, polyvinyl pyrrolidone, marine adhesive proteins, cyanoacrylates, analogs, mixtures, combinations or derivatives of the above. Alternatively, the matrix can be formed of naturally occurring biopolymers. Such naturally occurring biopolymers include, but are not limited to, fibrin, fibrinogen, fibronectin, collagen, or other suitable biopolymers. Also, the matrix can be formed from a mixture of naturally occurring biopolymers or synthetic polymers. Another example of a matrix material is an injectable citrate-based mussel-inspired bioadhesive (iCMBA).

The scaffold can include one or more matrix materials including, but not limited to, a collagen gel, a polyvinyl alcohol sponge, a poly(D,L-lactide-co-glycolide) fiber matrix, a polyglactin fiber, a calcium alginate gel, a polyglycolic acid mesh, polyester (e.g., poly-(L-lactic acid) or a polyanhydride), a polysaccharide (e.g. alginate), polyphosphazene, polyacrylate, or a polyethylene oxide-polypropylene glycol block copolymer. Matrices can be produced from proteins (e.g., extracellular matrix proteins such as fibrin, collagen, fibronectin), polymers (e.g., polyvinylpyrrolidone), or hyaluronic acid. Synthetic polymers can also be used, including bioerodible polymers (e.g., poly(lactide), poly(glycolic acid), poly(lactide-co-glycolide), poly(caprolactone), polycarbonates, polyamides, polyanhydrides, polyamino acids, polyortho esters, polyacetals, polycyanoacrylates), degradable polyurethanes, non-erodible polymers (e.g., polyacrylates, ethylene-vinyl acetate polymers or other acyl substituted cellulose acetates and derivatives thereof), non-erodible polyurethanes, polystyrenes, polyvinyl chloride, polyvinyl fluoride, poly(vinylimidazole), chlorosulphonated polyolifins, polyethylene oxide, polyvinyl alcohol, Teflon®, or nylon.

The scaffold can further comprise any other bioactive molecule, for example an antibiotic or an additional chemotactic growth factor or another osteogenic, dentinogenic, amelogenic, or cementogenic growth factor. In some embodiments, the scaffold is strengthened, through the addition of, e.g., human serum albumin (HSA), hydroxyethyl starch, dextran, or combinations thereof. Suitable concentrations of these compounds for use in the compositions of the application are known to those of skill in the art, or can be readily ascertained without undue experimentation.

The concentration of a compound or a composition in the scaffold can vary with the nature of the compound or composition, its physiological role, or desired therapeutic or diagnostic effect. A therapeutically effective amount is generally a sufficient concentration of therapeutic agent to display the desired effect without undue toxicity. For example, the matrix can include a composition comprising CTGF or TGFβ3 at any of the above described concentrations. The compound can be incorporated into the scaffold or matrix material by any known method. In some embodiments, the compound can be imbedded in a gel, e.g., a collagen gel incorporated into the pores of the scaffold or matrix material or applied as a coating over a portion, a substantial portion, substantially all of, or all of the scaffold or matrix material.

Alternatively, chemical modification methods can be used to covalently link a compound or a composition to a matrix material. The surface functional groups of the matrix can be coupled with reactive functional groups of a compound or a composition to form covalent bonds using coupling agents well known in the art such as aldehyde compounds, carbodiimides, and the like. Additionally, a spacer molecule can be used to gap the surface reactive groups and the reactive groups of the biomolecules to allow more flexibility of such molecules on the surface of the matrix. Other similar methods of attaching biomolecules to the interior or exterior of a matrix will be known to one of skill in the art.

Pores and channels of the scaffold can be engineered to be of various diameters. For example, the pores of the scaffold can have a diameter range from micrometers to millimeters. In some embodiments, the pores of the matrix material include microchannels. Microchannels generally have an average diameter of about 0.1 μm to about 1,000 μm, e.g., about 50 μm to about 500 μm (for example about 100 μm, 150 μm, about 200 μm, about 250 μm, about 300 μm, about 350 μm, about 400 μm, about 450 μm, about 500 μm, or about 550 μm). One skilled in the art will understand that the distribution of microchannel diameters can have any distribution including a normal distribution or a non-normal distribution. In some embodiments, microchannels are a naturally occurring feature of the matrix material(s). In other embodiments, microchannels are engineered to occur in the matrix materials.

Several methods can be used for fabrication of porous scaffolds, including particulate leaching, gas foaming, electrospinning, freeze drying, foaming of ceramic from slurry, and the formation of polymeric sponge. Other methods can be used for fabrication of porous scaffolds include computer aided design (CAD) and synthesizing the scaffold with a bioplotter (e.g., solid freeform fabrication) (e.g., Bioplotter™ EnvisionTec, Germany).

Biologic drugs that can be added to compositions and methods as described herein can include immunomodulators and other biological response modifiers. A biological response modifier generally encompasses a biomolecule (e.g., peptide, peptide fragment, polysaccharide, lipid, antibody) that is involved in modifying a biological response, such as the immune response or tissue or organ growth and repair, in a manner that enhances a particular desired therapeutic effect, for example, the cytolysis of bacterial cells or the growth of tissue- or organ-specific cells or vascularization. Biologic drugs can also be incorporated directly into the matrix component. Those of skill in the art will know, or can readily ascertain, other substances which can act as suitable non-biologic and biologic drugs.

Compositions described herein can also be modified to incorporate a diagnostic agent, such as a radiopaque agent. The presence of such agents can allow the physician to monitor the progression of wound healing occurring internally. Such compounds include barium sulfate as well as various organic compounds containing iodine. Examples of these latter compounds include iocetamic acid, iodipamide, iodoxamate meglumine, iopanoic acid, as well as diatrizoate derivatives, such as diatrizoate sodium. Other contrast agents that can be utilized in the compositions can be readily ascertained by those of skill in the art and can include, for example, the use of radiolabeled fatty acids or analogs thereof.

The concentration of an agent in the composition can vary with the nature of the compound, its physiological role, or desired therapeutic or diagnostic effect. A therapeutically effective amount is generally a sufficient concentration of therapeutic agent to display the desired effect without undue toxicity. A diagnostically effective amount is generally a concentration of diagnostic agent which is effective in allowing the monitoring of the integration of the tissue graft, while minimizing potential toxicity. In any event, the desired concentration in a particular instance for a particular compound is readily ascertainable by one of skill in the art.

Explant Model

An explant model, as that term is used herein, can be a well-established model for tissue damage or repair. An explant model can be a human explant model or non-human explant model, similar to a human meniscus. For example, an explant model can be mammal, reptile, or avian, more preferably, human, equine, bovine, rabbit, porcine, canine, cat, sheep, chicken, or goat. The non-human explant model can exhibit cross-reaction of human markers. qRT-PCR can be performed to design PCR primers and test for any species where the explant model has no cross reactivity with human markers.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations can contain a therapeutically effective amount of a biologically active agent described herein, which can be in purified form, together with a suitable amount of carrier so as to provide the form for proper administration to the subject.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, or rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) or reduce dosage frequency. Controlled-release preparations can also be used to effect the time of onset of action or other characteristics, such as blood levels of the agent, or consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating damaged or injured cartilaginous tissue in a subject in need thereof, so as to heal the damaged or injured cartilaginous tissue.

Scaffolds comprising compositions promoting recruitment of progenitor cells and differentiation into fibrochondrocyte-like cells, can be administered or implanted for treatment of fibrocartilage-related injuries, such as to the knee meniscus, intervertebral discs, temporomandibular joint (TMJ) ligaments, or tendons. For example, the sequential induction of stem cell recruitment, intermediate fibrous integration, or chondrogenic remodeling, can be administered for regeneration or healing of other cartilaginous tissues, including articular cartilage, temporomandibular joint (TMJ) disc, or ligament or tendon enthesis. The strategy can be applied to methods for treating tears reaching the avascular region (e.g., inner third zone) of the meniscus, pre-treatment for injury in the avascular region prior to meniscectomy to reduce the amount of meniscus to be removed, or a preventative treatment for patients at risk of osteoarthritis (OA). In some embodiments, the strategy can be used for a research tool for studying MSCs or fibrocartilaginous tissue development.

In some embodiments, the present disclosure can provide an approach to healing fibrocartilage-related injuries. For example, the present disclosure can provide an approach to healing a tear in fibrocartilage tissue, such as a longitudinal or vertical tear, a radial tear, a horizontal tear, a bucket handle tear, a parrot beak tear, or a flap tear.

In some embodiments, step-wise induction of stem cell recruitment, fibrogenic differentiation, and chondrogenic differentiation can serve as a novel and efficient strategy for seamless inner meniscus healing.

In some embodiments, seamless inner meniscus healing can not only shorten the time for return to work but can also reduce the incident rate of degenerative osteoarthritis. For example, the healing or regeneration by endogenous cell recruitment can improve healthcare for US military service members, given the extremely high incident rate of meniscus injuries in the populations.

Methods described herein can provide, in various embodiments, treatment for damaged avascular fibrocartilage tissues, which can be resistant to regeneration in the human body. As described herein, tissue such as fibrocartilage can be derived from progenitor cells such as mesenchymal stem cells (e.g., MSCs). Such derivation can occur in vitro or in vivo. The methods can use a mixture of growth factors to stimulate progenitor cells, such as MSCs, to differentiate into fibrochondrocytes or fibrochondrocyte-like cells, which can then be used as therapeutic cells to generate fibrocartilage. A fibrochondrocyte differentiation media can be used directly with progenitor cells in vitro or in vivo. A fibrochondrocyte differentiation media can be used in or on an acellular scaffold or a scaffold seeded with progenitor cells. Successful derivation of fibrocartilage can provide an option or supplement for repair treatments to tissues comprised of fibrocartilage.

Exemplary tissue for repair with compositions or methods described herein include, but are not limited to, damaged menisci (e.g., knee menisci), damaged ligaments, damaged tendons, damaged intervertebral discs, temporomandibular joints, or damaged triangular fibrocartilage. Damaged tissue can include defects such as tears, injuries, strain, sprain, pull, osteoarthritis, or degeneration.

In some embodiments, tissue for repair with compositions or methods described herein can include a meniscus. A meniscus is a crescent-shaped fibrocartilaginous structure that can partly divides a joint cavity. In humans, menisci can be present in the knee, wrist, acromioclavicular, sternoclavicular, and temporomandibular joints. In other organisms menisci can be present in other joints. The term ‘meniscus’ can be used to refer to the cartilage of the knee, either to the lateral or medial meniscus. The lateral or medial meniscus are cartilaginous tissues that provide structural integrity to the knee when it undergoes tension or torsion. The menisci can also be known as “semi-lunar” cartilages—referring to their half-moon, crescent shape.

The menisci of the knee consists of two pads of fibrocartilaginous tissue which serve to disperse friction in the knee joint between the lower leg (tibia) and the thigh (femur). Knee menisci are concave on the top and flat on the bottom, articulating with the tibia. While the ends of the thigh bone and the shin bone have a thin covering of soft hyaline cartilage, the menisci are made of tough fibrocartilage and conform to the surfaces of the bones they rest on. One meniscus rests on the medial tibial plateau; this is the medial meniscus. The other meniscus rests on the lateral tibial plateau; this is the lateral meniscus. Knee menisci are attached to the small depressions (fossae) between the condyles of the tibia (intercondyloid fossa), and towards the center they are unattached and their shape narrows to a thin shelf. The blood flow of the meniscus can be from the periphery (outside) to the central meniscus. Blood flow decreases with age and the central meniscus is avascular by adulthood leading to very poor healing rates.

The menisci can act to disperse the weight of the body and reduce friction during movement. Because the condyles of the femur and tibia meet at one point (which changes during flexion and extension), the menisci spread the load of the body's weight. Without the menisci, the weight of the body could be unevenly applied to the bones in the legs (the femur and tibia). This uneven weight distribution can cause the development of abnormal excessive forces leading to early damage of the knee joint. The menisci can also contribute to the stability of the joint. The menisci can be nourished by small blood vessels but have a large area in the center with no direct blood supply (avascular). This presents a problem when there is an injury to the meniscus, as the avascular areas tend not to heal. Without the essential nutrients supplied by blood vessels, healing cannot take place.

In some embodiments, tissue for repair with compositions or methods described herein can be caused by traumatic injury (often seen in athletes) or degenerative processes, which are the most common tear seen in all ages of patients. Meniscal tears can occur in all age groups. Traumatic tears are most common in active people aged 10-45. Traumatic tears are usually radial or vertical in the meniscus and more likely to produce a moveable fragment that can catch in the knee and therefore require surgical treatment.

In some embodiments, tissue for repair with compositions or methods described herein can be caused by an internally or externally rotated knee in a flexed position, with the foot in a flexed position. It is not uncommon for a meniscal tear to occur along with injuries to the anterior cruciate ligament ACL or the medial collateral ligament MCL—these three problems occurring together are known as the “unhappy triad,” which is seen in sports such as football when the player is hit on the outside of the knee. Subjects who experience a meniscal tear usually experience pain and swelling as their primary symptoms. Another common symptom is joint locking, or the inability to completely straighten the joint. This is due to a piece of the torn cartilage preventing the normal functioning of the knee joint.

In some embodiments, tissue for repair with compositions or methods described herein can include “torn cartilage”. Torn cartilage can refer to an injury to one of the menisci. There are two general types of meniscus injuries, acute tears that are often the result of trauma or a sports injury and chronic or wear-and-tear type tears. Acute tears have many different shapes (vertical, horizontal, radial, oblique, complex) and sizes. They can be treated with surgical repair depending upon the patient's age as they rarely heal on their own. Chronic tears can be treated symptomatically: physical therapy with or without the addition of injections and anti-inflammatory medications. If the tear causes continued pain, swelling, or knee dysfunction, then the tear can be removed or repaired surgically.

In some embodiments, tissue for repair with compositions or methods described herein can include a tear of a meniscus. A tear of a meniscus can include the rupturing of one or more of the fibrocartilage strips in the knee called menisci. “Torn cartilage” in the knee can refer to an injury to a meniscus at the top of one of the tibiae. Menisci can be torn during innocuous activities such as walking or squatting. They can also be torn by traumatic force encountered in sports or other forms of physical exertion. The traumatic action is most often a twisting movement at the knee while the leg is bent. In older adults, the meniscus can be damaged following prolonged ‘wear and tear’ called a degenerative tear. Tears can lead to pain or swelling of the knee joint. Especially acute injuries (typically in younger, more active patients) can lead to displaced tears which can cause mechanical symptoms such as clicking, catching, or locking during motion of the knee joint. The joint will be in pain when in use, but when there is no load, the pain goes away.

In some embodiments, tissue for repair with compositions or methods described herein can include the “unhappy triad” is a set of commonly co-occurring knee injuries which includes injury to the medial meniscus. A tear of the lateral meniscus can occur as part of the unhappy triad, together with a tear of the anterior cruciate ligament and medial collateral ligament.

In some embodiments, tissue for repair with compositions or methods described herein can be a degenerative tear. Degenerative tears are most common in people from age 40 upward but can be found at any age, especially with obesity. Degenerative meniscal tears are thought to occur as part of the aging process when the collagen fibers within the meniscus start to break down and lend less support to the structure of the meniscus. Degenerative tears can be horizontal, producing both an upper and a lower segment of the meniscus. These segments do not usually move out of place and can be less likely to produce mechanical symptoms of catching or locking.

In some embodiments, meniscal tear for repair with compositions or methods described herein can be classified in various ways. For example, meniscal tears can be classified by anatomic location, by proximity to blood supply, etc. meniscal tear for repair with compositions or methods described herein can be characterized by tear patterns, including radial tears; flap or parrot-beak tears; peripheral tears; longitudinal tears; bucket-handle tears; horizontal cleavage tears; or complex, degenerative tears. For example, these tears can then be further classified by their proximity to the meniscus blood supply, namely whether they are located in the “red-red,” “red-white,” or “white-white” (e.g., avascular zone, inner region, inner third zone) zones.

In some embodiments, the compositions and methods described herein can be used to treat any of the above meniscus defects or injuries.

In some embodiments, compositions described herein can be administered (e.g., through injection) between intervertebral discs to prevent or treat disc degeneration. As another example, compositions described herein can be administered to prevent or treat arthritis.

In some embodiments, the compositions and methods described herein can be used to treat any of the above cartilage-related defects or injuries.

Various embodiments provide compositions and methods to recruit, home, or induce differentiation of progenitor cells by using a cell homing composition and subsequently promote or induce differentiation of recruited progenitor cells to form fibrochondrocyte or fibrochondrocyte-like cells using composition comprising CTGF or TGFβ3. A cell homing composition, a chemotactic, profibrogenic, chondrogenic composition, or a scaffold or matrix can be implanted in a tissue defect of a subject so as to recruit endogenous progenitor cells into the scaffold or matrix material and differentiate recruited progenitor cells to fibrochondrocyte or fibrochondrocyte-like cells.

In some embodiments, methods of causing progenitor cells to migrate to a scaffold and differentiate to form fibrochondrocyte or fibrochondrocyte-like cells in the scaffold are provided. The method can include placing a scaffold containing a cell homing composition and a chemotactic, profibrogenic, or chondrogenic composition in fluid communication with cells. As used herein, a scaffold is in “fluid communication” with a cell if the cell has no physical barrier (e.g., a basement membrane, areolar connective tissue, adipose connective tissue, etc.) preventing the cell from migrating to the scaffold. Without being bound to any particular mechanism, it is believed that the cell migrates to the scaffold along a moist path from its source, in response to the presence of a cell homing composition forming a concentration gradient to the cell, and thereby influencing the cell to migrate toward the higher concentrations of the cell homing composition in the scaffold.

The scaffold optionally does not comprise a transplanted mammalian cell, i.e., no cell is applied to the scaffold; any cell present in the scaffold migrated into the scaffold. A scaffold is generally understood to be a three-dimensional structure into which cells, tissue, vessels, etc., can grow, colonize or populate when the scaffold is placed into a tissue site. A scaffold of the method can be as discussed herein.

The compositions and methods described herein hold significant clinical value because of their ability to recruit endogenous progenitor cells, thereby optionally avoiding transplant of cells to a subject.

Methods described herein are generally performed on a subject in need thereof. A subject in need of the therapeutic methods described herein can be a subject having, diagnosed with, suspected of having, or at risk for developing cartilage-related degeneration, injury, or damage or osteoarthritis (OA). A determination of the need for treatment will typically be assessed by a history and physical exam consistent with the disease or condition at issue. Diagnosis of the various conditions treatable by the methods described herein is within the skill of the art. The subject can be an animal subject, preferably a mammal, more preferably horses, cows, dogs, cats, sheep, pigs, mice, rats, monkeys, guinea pigs, or chickens, or most preferably a human.

An effective amount of composition including one or more of progenitor cells, fibrochondrocytes, fibrochondrocyte-like cells, chemotactic factor, profibrogenic factor, chondrogenic factor, CTGF, TGFβ3, or fibrocartilage described herein is generally that which can restore, at least partially or fully, structure or function to the tissue of interest. An effective amount of a scaffold comprising a composition including one or more of progenitor cells, fibrochondrocytes, fibrochondrocyte-like cells, chemotactic factor, profibrogenic factor, chondrogenic factor, CTGF, TGFβ3, or fibrocartilage described herein is generally that which can restore, at least partially or fully, structure or function to the tissue of interest.

In various embodiments, an effective amount of chemotactic or profibrogenic factor described herein can substantially improve the formation of fibrous integration.

In various embodiments, an effective amount of a chondrogenic factor described herein can substantially improve the remodeling of the fibrous matrix to cartilaginous tissue by inducing chondrogenic differentiation.

Damaged or injured (e.g., torn) cartilage tissue can be repaired using a chemotactic or profibrogenic factor followed by a chondrogenic factor. Torn cartilage tissue can be repaired using substrates (i.e., matrix materials) comprising compositions promoting recruitment of progenitor cells and differentiation into fibrochondrocyte-like cells.

An effective amount of a cell homing composition (e.g., chemotactic or profibrogenic factors) comprising, for example, CTGF can be that which can induce recruitment of progenitor cells or migration of progenitor cells. An effective amount of a chondrogenic composition can be that which can induce differentiation of progenitor cells to fibrochondrocytes or fibrochondrocyte-like cells. An effective amount of a scaffold or matrix material containing cell homing composition and a chondrogenic composition can be that which can induce recruitment of progenitor cells or migration of progenitor cells and induce differentiation of recruited progenitor cells to fibrochondrocytes or fibrochondrocyte-like cells. An effective amount of a scaffold or matrix material containing a cell homing composition and a fibrochondrogenic composition can be that which can recruit and induce migration of a sufficient number of progenitor cells and induce at least a portion of recruited progenitor cells to form a fibrochondrocytes or fibrochondrocyte-like cells so as to increase biological function of a tissue or organ. An effective amount of a scaffold or matrix material containing cell homing composition and a fibrochondrogenic composition can be that which restores function or appearance to damaged, injured, or torn tissue comprising some combination of cartilage, tendon, ligament, or bone.

As an example, a subject in need can have a fibrocartilage cell or tissue deficiency of at least about 5%, about 10%, about 25%, about 50%, about 75%, about 90% or more, and compositions and methods described herein can provide an increase in number or function of fibrochondrocytes cells or fibrocartilage tissues. As another example, a subject in need can have damage to a tissue or organ, and the method can provide an increase in biological function of the tissue or organ by at least about 5%, about 10%, about 25%, about 50%, about 75%, about 90%, about 100%, or about 200%, or even by as much as about 300%, about 400%, or about 500%. As yet another example, the subject in need can have an fibrocartilage-related disease, disorder, or condition, and the method provides an engineered scaffold sufficient that can recruit progenitor cells and form fibrocartilage cells or tissue sufficient to ameliorate or stabilize the disease, disorder, or condition. For example, the subject can have a disease, disorder, or condition that results in the loss, atrophy, dysfunction, or death of fibrocartilage cells. In a further example, the subject in need can have an increased risk of developing a disease, disorder, or condition that is delayed or prevented by the method. As yet another example, the subject in need can have experienced death or dysfunction of fibrocartilage cells as the result of a side effect of a medication used for the treatment of another disease or disorder.

Implantation of an engineered construct is within the skill of the art. The scaffold or matrix material can be either fully or partially implanted into a tissue or organ of the subject to become a functioning part thereof. Preferably, the implant initially attaches to and communicates with the host through a cellular monolayer. Over time, endogenous cells can migrate into the scaffold to form tissue. The cells surrounding the engineered tissue can be attracted by biologically active materials, including biological response modifiers, such as polysaccharides, proteins, peptides, genes, antigens, or antibodies, which can be selectively incorporated into the matrix to provide the needed selectivity, for example, to tether the cell receptors to the matrix, stimulate cell migration into the matrix, or both. The matrix can comprise a gelled phase and interconnecting channels that allow for cell migration, augmented by both biological and physical-chemical gradients. For example, cells surrounding the implanted matrix can be attracted by biologically active materials including CTGF or TGFβ3. One of skill in the art will recognize and know how to use other biologically active materials that are appropriate for attracting cells to the matrix.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of composition including one or more of progenitor cells, fibrochondrocytes, fibrochondrocyte-like cells, chemotactic factor, profibrogenic factor, chondrogenic factor, CTGF, TGFβ3, or fibrocartilage can be employed in pure form or, where such forms exist, in pharmaceutically acceptable salt form and with or without a pharmaceutically acceptable excipient. For example, the compounds as described herein can be administered, at a reasonable benefit/risk ratio applicable to any medical treatment, in a sufficient amount to restore, at least partially or fully, structure or function to the tissue of interest.

The amount of a composition described herein that can be combined with a pharmaceutically acceptable carrier to produce a single dosage form can vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where large therapeutic indices are preferred.

The specific therapeutically effective dose level for any particular subject can depend upon a variety of factors including the injury or disorder being treated and the severity of the injury or disorder; activity of the specific compound employed; the specific composition employed; the age, body weight, general health, sex and diet of the patient; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Shawl (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions as described herein will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of compositions or scaffold comprising compositions described herein can occur as a single event or over a time course of treatment. For example, a composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for fibrocartilage-related degeneration or injury.

Various compositions described herein can be administered simultaneously or sequentially with another agent, such as an antibiotic, an anti-inflammatory, or another agent. For example, a composition including one or more of progenitor cells, fibrochondrocytes, fibrochondrocyte-like cells, chemotactic factor, profibrogenic factor, chondrogenic factor, CTGF, TGFβ3, or fibrocartilage can be administered simultaneously with another agent, such as an antibiotic or an anti-inflammatory. Simultaneous administration can occur through administration of separate compositions, each containing one or more of a composition described herein, an antibiotic, an anti-inflammatory, or another agent. Simultaneous administration can occur through administration of one composition containing two or more of progenitor cells, fibrochondrocytes, fibrochondrocyte-like cells, chemotactic factor, profibrogenic factor, chondrogenic factor, CTGF, TGFβ3, fibrocartilage, an antibiotic, an anti-inflammatory, or another agent. Compositions described herein can be administered sequentially with an antibiotic, an anti-inflammatory, or another agent. For example, a composition described herein can be administered before or after administration of an antibiotic, an anti-inflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels, injectable hydrogels, hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, or copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres (μS), hydrogels, polymeric implants, smart polymeric carriers, or liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to progenitor cells, fibrochondrocytes, fibrochondrocyte-like cells, chemotactic factor, profibrogenic factor, chondrogenic factor, CTGF, TGFβ3, culture medium, induction supplement, or fibrocartilage. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Step-Wise Fibrochondrogenic Differentiation of Mesenchymal Stem/Progenitor Cells

The following example shows that treatment with a profibrogenic factor for a limited duration, followed by chondrogenic stimulation can induce step-wise differentiation of bone marrow or synovium MSCs into fibrochondrocyte-like cells.

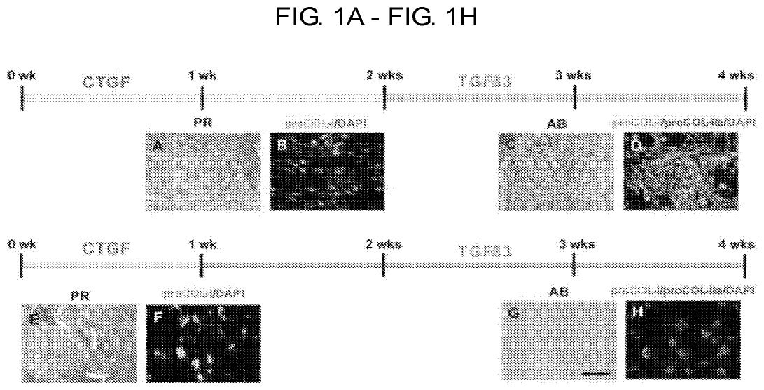

More specifically, the following example shows a novel strategy to induce seamless healing of inner meniscus repairs (see e.g., FIG. 1). Chemotaxis or other profibrogenic factors (e.g., CTGF) are applied to the torn meniscus via fibrin glue to recruit synovium MSCs into the torn site, followed by formation of fibrous integration. Then the intermediate fibrous matrix is remodeled to cartilaginous tissue by inducing chondrogenic differentiation with growth factor treatment (e.g., TGF-β3). Consistent in meniscus development, the following example demonstrates premature COL-I rich fibrous tissue (E16 & P8w) differentiates into fibrocartilage (Pazin+ JOR 2014) (see e.g., FIG. 1).

Embryonic (E16) menisci and young (8 weeks old) menisci are presently thought to be immature fibrous tissues predominantly expressing collagen type I that gradually transform into zone-specific fibrocartilage with increased expressions of collagen type II and aggrecan by 9 months-old.

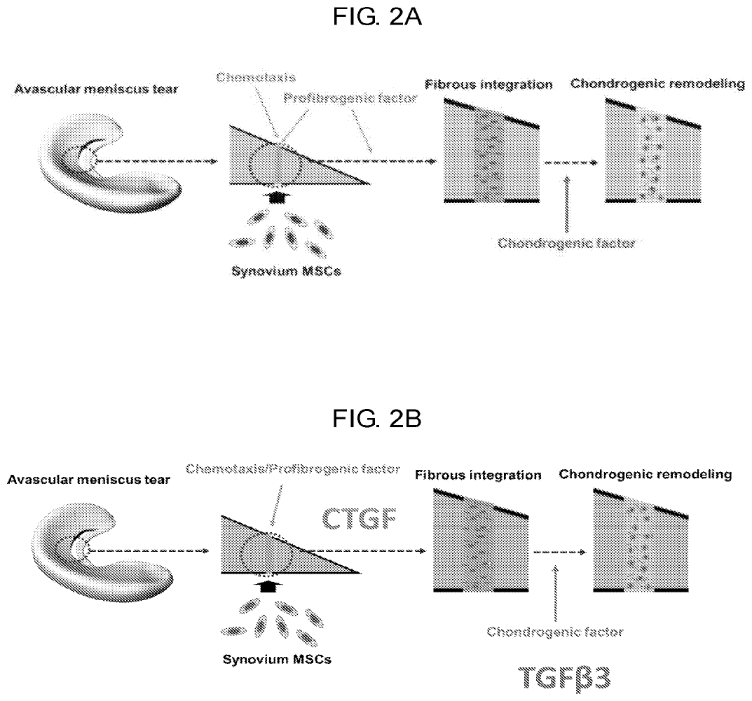

While attempting to derive fibrochondrocytes from mesenchymal stem/progenitor cells (MSCs), it was discovered that treatment with a profibrogenic factor for a limited duration, followed by chondrogenic stimulation can induce step-wise differentiation of bone marrow or synovium MSCs into fibrochondrocyte-like cells (see e.g., FIG. 1). Accordingly, the following experiments test a novel strategy to induce healing of inner meniscus tears that involves sequentially controlled (1) stem/progenitor cell recruitment; (2) formation of integrated intermediate fibrous matrix; and (3) cartilaginous remodeling into zone-specific fibrocartilage (see e.g., FIG. 2).

### Example 2: Optimize Parameter Determination for Multiple Cytokines/Growth Factors

The following example determines optimal doses, sequence, and duration of multiple cytokines/growth factors to induce temporally controlled stem cell recruitment and step-wise differentiation for inner meniscus healing in vitro.

Various delivery strategies were applied, including local delivery, controlled release, and exogenous treatment, to find the optimal doses, sequence, and duration of each growth factor to lead to the seamless healing of inner meniscus tears.

It is presently thought that the stem/progenitor cells giving rise to meniscus fibrochondrocytes follow a step-wise differentiation: intermediate fibrogenic differentiation followed by chondrogenic differentiation (see e.g., FIG. 1). Given that collagenous fibrous matrix is the default filler for tissue repair, integration and remodeling, the induction of intermediate fibrous matrix in the torn meniscus is presently thought to provide initial integrated filler which are remodeled to cartilaginous tissue. It was discovered that a profibrogenic treatment in MSCs longer than 2 weeks, followed by chondrogenic treatment failed to result in cartilaginous matrix (see e.g., FIG. 1). The 1 week profibrogenic treatment (i.e., CTGF), followed by a 3 week chondrogenic treatment (i.e., TGFβ3) yielded intermediate fibrous matrix at 1 week and cartilaginous matrix at 4 weeks (see e.g., FIG. 1). Likewise, it is presently thought that too short period of profibrogenic treatment could be insufficient to initiate tissue remodeling and integration. In addition to the treatment duration, the functions of growth factors frequently vary depending upon doses. Accordingly, the following optimizes duration and doses of growth factors for an efficient induction of step-wise stem cell recruitment, tissue integration, and differentiation that lead to seamless healing of avascular meniscus tears.

Multiple cytokines/growth factors reported as chemotaxis, profibrogenic, and chondrogenic stimulants are tested to find the best performers for inner meniscus healing. Given the reliability and proven functionality on in vitro meniscus healing, a well-established meniscus explant model is utilized to find optimal conditions for avascular meniscus healing.

Experimental Design and Data Analysis.

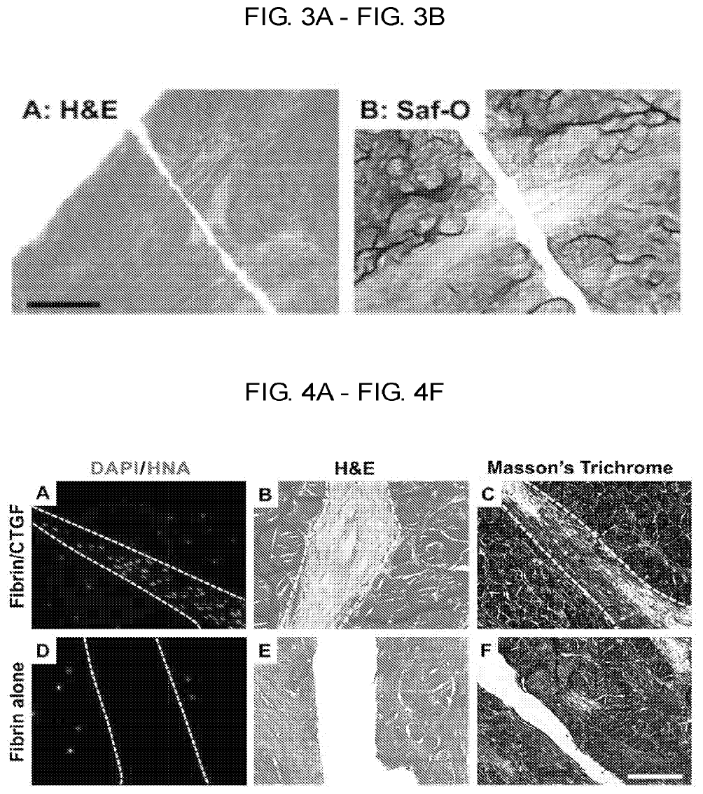

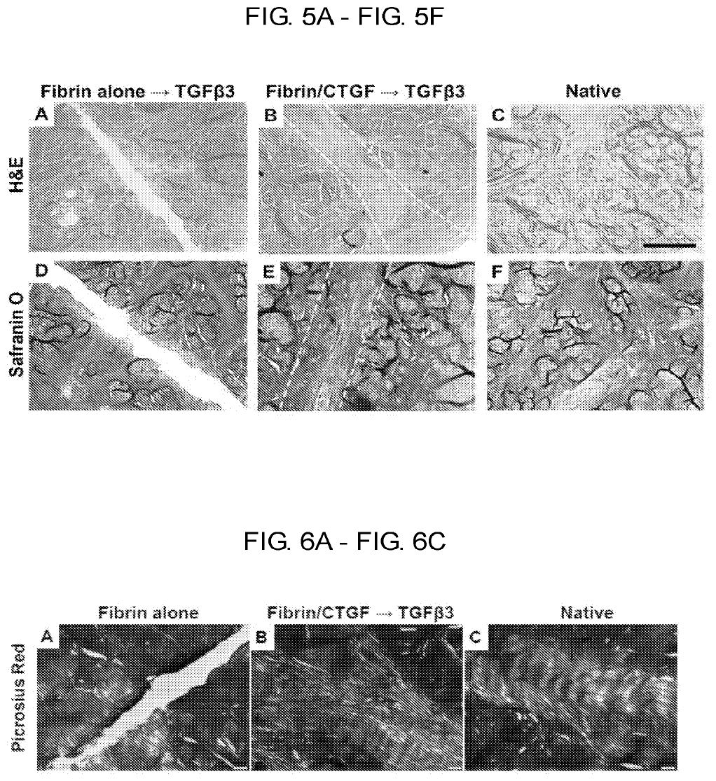

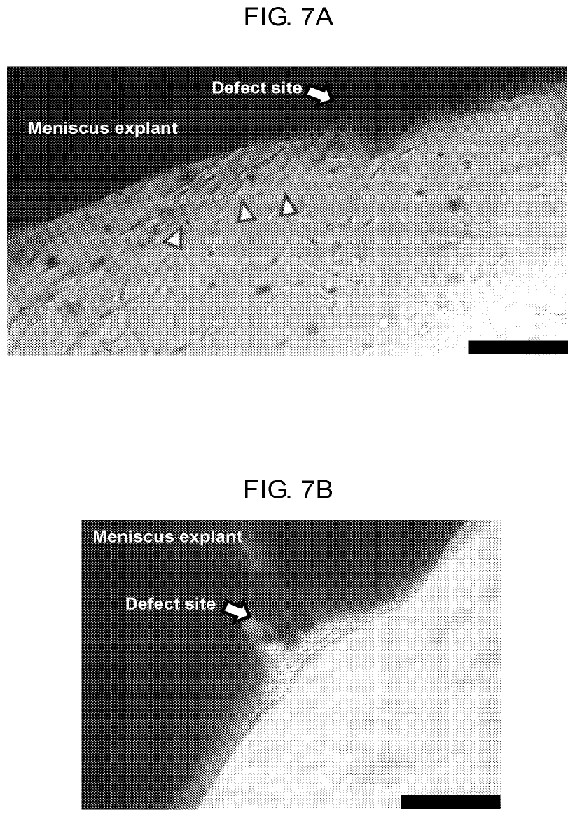

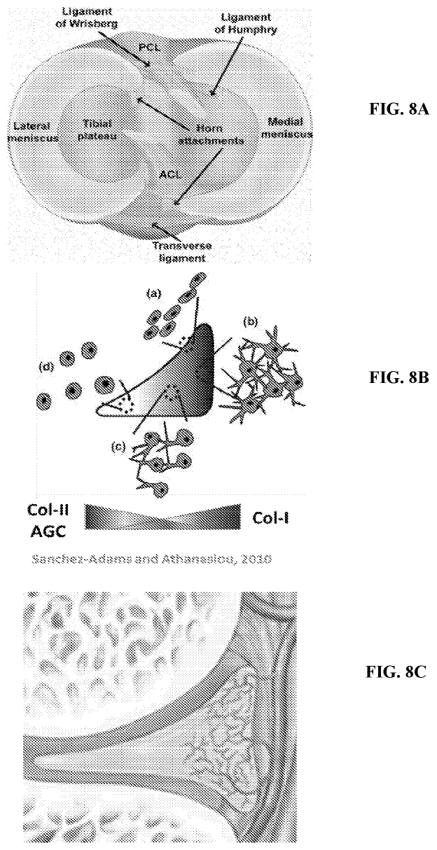

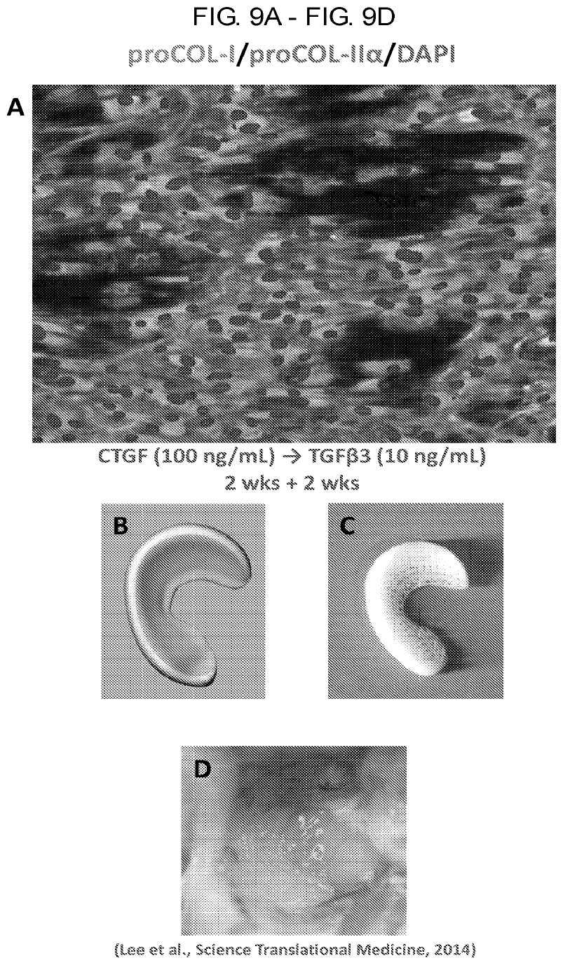

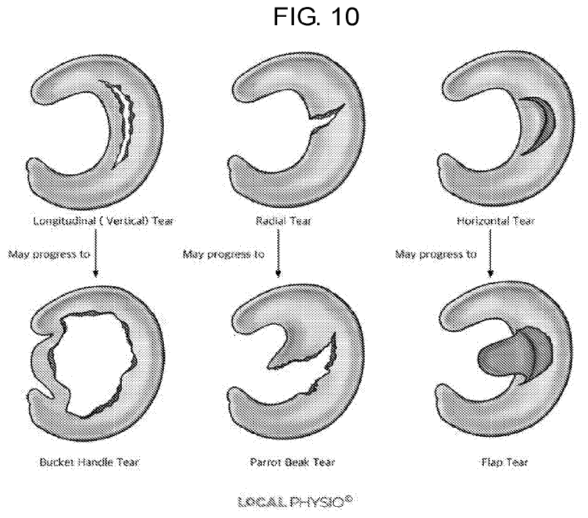

A well-established meniscus explant model was used to study in vitro healing of avascular meniscus tears. Menisci were isolated from skeletally mature bovine knee joints provided by a local butcher shop. Upon creation of a longitudinal tear using a surgical blade in the inner third zone, fibrin glue is applied to the torn site. Briefly, 50 mg/mL fibrinogen and 50 U/mL thrombin is co-injected in between the incised tissue surfaces using FibriJet® dual-injector with a blending applicator (Nordson Micromedics, Westlake, Ohio). Example 2 and Example 4 showed that tears in inner third zone of bovine menisci, with or without fibrin glue, failed to heal or integrate by 6 weeks culture in vitro (see e.g., FIG. 3).

To induce stem cell migration and formation of intermediate fibrous integration, a selected chemotaxis and/or profibrogenic cue is delivered through the fibrin gel or applied exogenously for a selected duration. To remodel the temporary fibrous matrix into cartilaginous tissue, chondrogenic cues are applied up to 6 weeks afterward. The list of cytokines/growth factors, mode and duration of each treatment, and harvest time points are summarized in TABLE 1. The meniscus explants are placed on top of monolayer-cultured P2-3 human synovium mesenchymal stem/progenitor cells (MSCs) at 80-90% confluence. To track the cell migration and engraftment, MSCs are labeled with Vybrant® CFDA (Life technologies, Grand Island, N.Y.). Supplements for fibrogenic and chondrogenic differentiation, are applied along with profibrogenic and chondrogenic factors, respectively.

TABLE 1
List of chemotaxis, profibrogenic, and chondrogenic cues for avascular meniscus healing.

| | Chemotaxis | Profibrogenic cues | Chrondrogenic cues |
|---|---|---|---|
| Cytokines/ | SDF-1, FGF-2, CTGF | FGF-2, CTGF, GDF-5 | TGFβ1, TGFβ3, IFG-1, BMP7 |
| growth factors | | | |
| Dose | 50, 100, 200, 500 ng/mL | 10, 100, 1000 ng/mL | 10, 50, 100 ng/mL |
| Delivery mode | Fibrin gel | Fibrin gel; Exogenous | Exogenous |
| Duration | Release from fibrin gel in | Release from fibrin gel in ~5 | Exogenous treatment for 2, 3, |
| | ~5 days (data not shown) | days; Exogenous treatment | 4, 5 and 6 wks |
| | | for 3, 7, 10 or 14 days | |

Given the multiple testing conditions for the step-wise induction of inner meniscus healing, outcome in each step is analyzed using relatively straightforward methods as described in TABLE 2. In-depth characterization of healed meniscus tissue is performed in Example 3.

TABLE 2
Qualitative and quantitative analyses for step-wise inner meniscus healing.

| | | | Cartilaginous tissue |
|---|---|---|---|
| | Cell migration and | | remodeling and |
| Variables | engraftment | Intermediate fibrous integration | integration |
| Time | 1, 3, 5 and 7 days | 3, 7, 10 and 14 days | 2, 3, 4, 5 and 6 wks of |
| points | | | chondrogenic treatment |
| Methods | Tracking CFDA⁺ cells; | H&E, picrosirious red with polarized | H&E, Alcian blue, Safranin- |
| | immunofluorescence | image, Masson's trichrome | O, Masson's trichrome |
| Success | Labeled cells migrated in | Collagenous fibrous tissue | Seamless tissue integration |
| criteria | the defect site and | formation and integration | with cartilaginous matrix |
| | engrafted in fibrin matrix | | |

### Example 3: Characterize Tissue and Cellular Phenotypes in the Healed Meniscus

The following examples characterize tissue and cellular phenotypes in the healed meniscus using histology, qRT-PCR, nano-indentation, computerized histomorphometry, and immunohistochemistry.

Previous studies have rarely characterized healed meniscus tissues thoroughly and completely. It is presently thought that chondrocyte-like cell population and cartilaginous extracellular matrix are significant for long-term stability and functions in inner meniscus healing. This study shows avascular zone-specific distribution of extracellular matrix in the native meniscus can be recapitulated in a healed meniscus. This Example describes in-depth characterization of cell and tissue phenotypes in the healed meniscus tissue using multi-level analysis methods. Given the importance of mechanically stable integration for functional restoration of meniscus, pull-out tests are performed to analyze the integration strength, and nanoindentation are utilized to characterize mechanical properties of healed tissue given its narrow width (˜100 μm).

Experimental Design and Data Analysis.

Meniscus explants with artificially created inner zone tears are treated by a sequence of growth factors as in Example 2. Upon harvest, healed meniscal tissues are qualitatively and quantitatively analyzed as summarized in TABLE 3.

TABLE 3
Qualitative and quantitative analyses of healed meniscal tissues.

| | Histology and | Immunohistochemistry and | |
|---|---|---|---|
| Groups | histomorphometry | biochemical assays | Mechanical Properties |
| Variables | Quality of meniscus healing, | Inner zone-specific matrix | Pull-out strength, |
| | tissue integration, matrix | markers: COL-II, GAG, AGC, | viscoelastic properties |
| | remodeling; inner zone- | COL-IV, COL-I (control) | |
| | specific cell morphology | | |
| Methods | H&E, picrosirious red with | Qualitative: Monoclonal and | Pull-out test at constant |
| | polarized image, Masson's | polyclonal atibodies^{5-7, 10-} | displacement rate, |
| | trichrome, Alcian blue, | Quantitative: Biochemical assays | nanoindentation for |
| | Safranin-O | of COL-II and GAG; qRT-PCR for | viscoelastic properties |
| | | gene expressions; Odyssey infra- | (creep and stress-relaxation |
| | | red imaging | and modulus mapping |
| Success | Cartilaginous tissue | Formation of inner zone-specific | Mechanical properties within |
| criteria | formation; Chondrocyte-like | matrices quantitatively | 80% of the native avascular |
| | rounded cells; Seamless | approximate native tissues | meniscus tissues |
| | healing and integration | | |

Pull-Out Tests.

Samples for the pull-out tests are prepared using a cryotome as 500˜600 μm in thickness and a width of 1 mm. Upon mounting with tensile jigs in an isotonic saline bath at RT, a 0.02-N tare load is applied to the samples and then the samples are elongated at 10%/min until failure. The maximum force is obtained from the force vs. elongation curve. All pull-out tests are performed using Electroforce® BioDynamics® system (Bose Corp., Eden Prairie, Minn.). Nanoindentation: Nanoindentation experiments are conducted using a PIUMA™ nano-indenter (Opticsl 1, Amsterdam, The Netherlands). Briefly, a probe of 1-μm radius is used for the indentation experiments, with the sample loaded to a maximum force of 10 mN. All nanoindentation experiments were carried out on unfixed and unstained tissue sections. A series of indentations are performed to determine the variation in mechanical properties across a healed region, using the embedded high-precision X-Y stage with a 1-μm resolution. The drift rate is applied for stress-relaxation or creep.

### Example 4: Seamless Healing of Inner Meniscus Tears

A well-established meniscus explant model was used to study in vitro healing of avascular meniscus tears by temporal control of stem cell recruitment and step-wise fibrocartilaginous differentiation. Menisci were isolated from skeletally mature bovine knee joints provided by local butcher shop. A total of 4-5 wedge-shaped explants were prepared from a meniscus by cutting it in radial direction. Upon creation of a an incision (e.g., longitudinal or circumferential) using a surgical blade in the inner third zone (i.e., avascular zone), fibrin glue was applied to the incised site. Briefly, 50 mg/mL fibrinogen and 50 U/mL thrombin with or without 100 ng/mL connective tissue growth factor (CTGF) were co-injected in between the incised tissue surfaces using FibriJet® dual-injector with a blending applicator (Nordson Micromedics, Westlake, Ohio). Then the meniscus explants were cultured on top of monolayer-cultured human synovium-derived MSCs, as the potential cell source for meniscus regeneration in vivo. After 10 days, 10 ng/mL transforming growth factor beta 3 (TGFβ3) was applied with chondrogenic supplements. At day 10 and 6 weeks, explants were harvested, fixed in formalin, and sectioned for histological analysis. Recruited human MSCs were identified by immunolabeling human nucleus antigen (see e.g., FIG. 4). The concentration of CTGF and fibrin gel and the timeline for each treatment were pre-optimized by a pilot study (data not shown).

CTGF delivery via fibrin glue successfully recruited synovium MSCs into the incised site of inner meniscus by day 10 (see e.g., FIG. 3A). In vitro release study demonstrated that 90% of CTGF loaded in fibrin gel was released in 6 days. H&E and Masson's Trichrome staining demonstrated that integrated fibrous matrix was formed in the healing region by day 10 with CTGF-loaded fibrin. In contrast, the incised meniscus glued with fibrin alone failed to recruit MSCs or to induce any noticeable tissue integration (see e.g., FIG. 3D-3F). Upon TGFβ3 treatment from day 10, by 6 weeks the intermediate fibrous matrix was successfully remodeled into fibrocartilaginous matrix, fully integrating incised meniscal tissues (FIG. 3B, FIG. 3E). The healed meniscal tissues showed native-like fibrocartilaginous phenotype with rounded chondrocyte-like cells (see e.g., FIG. 4B, FIG. 4C, FIG. 4F). However, fibrin glue alone, followed by TGFβ3 treatment failed to form tissue integration or remodeling (see e.g., FIG. 4A, FIG. 4D).

These findings demonstrate a novel strategy to induce seamless healing of avascular inner meniscus tears. Transient delivery of CTGF via fibrin gel successfully recruited synovial MSCs into defect site and formed intermediate fibrous integration within 10 days. The initial fibrous matrix in healing region was then successfully remodeled into native-like fibrocartilaginous tissue upon 6 weeks TGFβ3 treatment. It was determined that a short-term delivery of CTGF for 1-4 days was not sufficient to induce fibrous tissue integration, whereas a prolonged CTGF application over 10 days attenuated fibrocartilaginous remodeling after TGFβ3 treatment (data not shown). Accordingly, the optimal duration and dose of CTGF selected in this study can be significant in providing initial fibrous integration that is readily remodeled into fibrocartilaginous tissue. As shown herein, guidance is provided for direct translation to in vivo applications for inner meniscus healing and development of control-delivery system to provide sequential/sustained release of CTGF and TGFβ3. In conclusion, the strategy to induce seamless healing of inner meniscus tears by temporal control of stem cell recruitment and differentiation serves as an efficient and directly translational approach to overcome the current limitations of meniscus repair.

The above examples show that a timely controlled application of connective tissue growth factor (CTGF) and TGFβ3 successfully improves healing of avascular meniscus tears by inducing recruitment and step-wise fibrocartilaginous differentiation of mesenchymal stem/progenitor cells (MSCs). It demonstrates that durations of CTGF and TGFβ3 are important factors for the timely regulated MSC recruitment, formation intermediate fibrous integration, and fibrocartilaginous remodeling. It was also shown that the novel strategy inducing inner meniscus healing can overcome limitations of the current treatments of meniscus injuries.

### Example 5: Avascular Meniscus Healing with CTGF-Loaded Fibrin Glue Mixed with Sustained Release TGFβ3 PLGA Microspheres (μS)

The following example shows that treatment with CTGF-loaded fibrin glue mixed with sustained release TGFβ3 PLGA microspheres (μS) can induce seamless healing of avascular inner meniscus tears by stem cell recruitment.

This example shows the application of the timely controlled delivery of CTGF and TGFβ3 for avascular meniscus healing by a single application of CTGF-loaded fibrin glue mixed with PLGA microspheres (μS)-encapsulating TGFβ3 in the explant model. This approach provides a minimally invasive delivery of CTGF in a short-term and TGFβ3 for a prolonged period for in vivo applications.

Further, this example shows that the short-term release of CTGF from fibrin glue and the sustained release of TGFβ3 from PLGA μS can lead to seamless healing of avascular meniscus tears by regulating timely controlled recruitment and differentiation of synovial MSCs.

Methods.

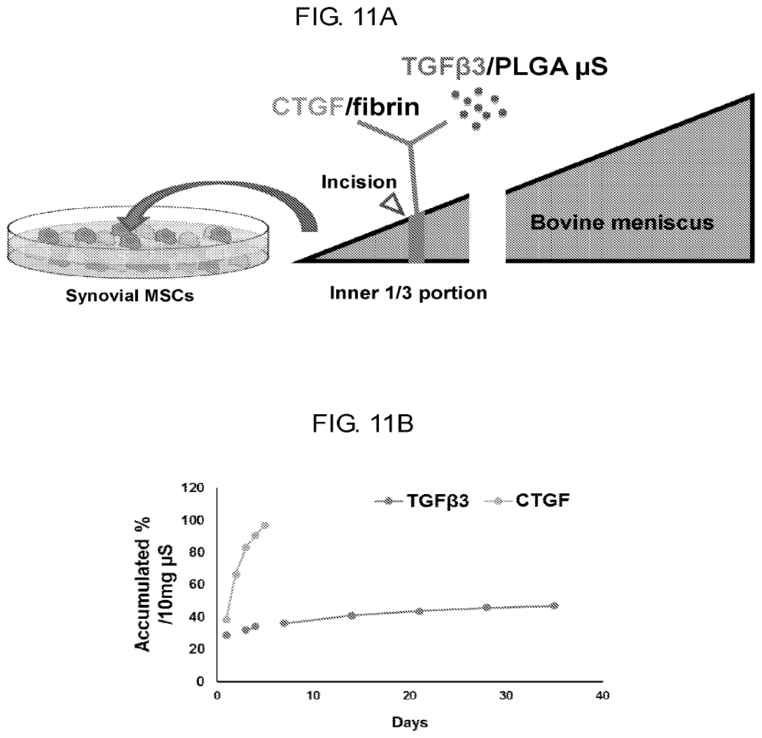

A modified meniscus explant model was used to study in vitro healing of avascular meniscus tears. Menisci were isolated from skeletally mature bovine knee joints provided by a local butcher shop. As illustrated in FIG. 11A, total 4-5 wedge-shaped explants were prepared from a meniscus by cutting it in radial direction. Upon creation of a longitudinal incision using a surgical blade in the inner third zone, fibrin glue loaded with 100 ng/ml CTGF and TGFβ3-μS was applied to the incised site. Briefly, 50 mg/mL fibrinogen and 50 U/mL thrombin with or without 100 ng/mL connective tissue growth factor (CTGF) and 10 mg of PLGA μS-encapsulating TGFβ3 were co-injected in between the incised tissue surfaces using FibriJet® dual-injector with a blending applicator (Nordson Micromedics, Westlake, Ohio). PLGA μS-encapsulating TGFβ3 (2.5 μg per 250 mg PLGA 50:50) were prepared by the double-emulsion technique, and in vitro release kinetics of CTGF and TGFβ3 were measured by ELISA up to 35 days. The meniscus explants were then cultured on top of monolayer-cultured human synovium-derived MSCs for 6 weeks with fibrous and/or chondrogenic supplements. At 10 days, cell recruitment and intermediate fibrous integration was confirmed or analyzed by histology and labeling human nucleus antigen. At 6 weeks, all explants were harvested and analyzed for healing of avascular tears using histology and mechanical testing.

Results.

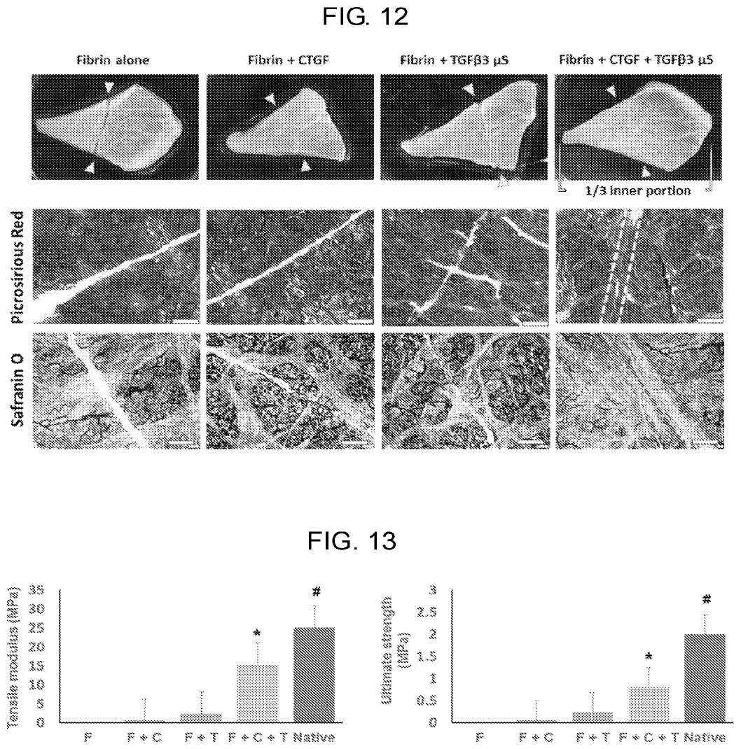

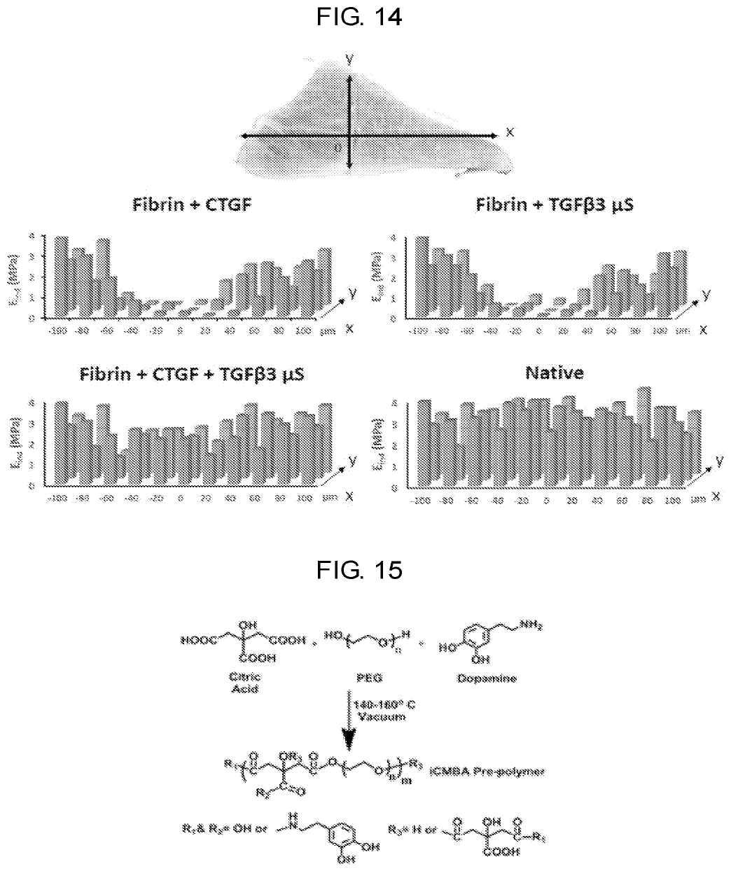

CTGF delivery via fibrin glue successfully recruited synovium MSCs into the incised site of inner meniscus by day 10 (data not shown). In vitro release study demonstrated a short-term release of CTGF within 6 days and sustained release of TGFβ3 over 35 days (see e.g., FIG. 11B). Macroscopic images and histology showed CTGF-loaded fibrin glue with TGFβ3-μS successfully led to seamless healing of avascular meniscus incision, whereas fibrin alone, fibrin with CTGF, and fibrin with TGFβ3-μS failed to lead to integration of the torn meniscus tissues (see e.g., FIG. 12). Consistently, Picrosirious Red (PR) and Safranin 0 (Saf-O) staining demonstrated that CTGF-loaded fibrin glue with TGFβ3-μS resulted in native-like fibrocartilaginous healing of meniscus incision, as compared to the remained gaps with fibrin alone, fibrin with CTGF, and fibrin with TGFβ3-μS (see e.g., FIG. 12). Tensile stiffness and ultimate strength of the healed tissue significantly improved after 6 weeks with CTGF-loaded fibrin glue+TGFβ3-μS, in comparison with all other groups (see e.g., FIG. 13).

### SUMMARY AND DISCUSSION

These findings demonstrate a novel strategy to induce seamless healing of avascular inner meniscus tears by stem cell recruitment. Transient delivery of CTGF via fibrin gel and sustained release of TGFβ3 from PLGA μS successfully recruited synovial MSCs into the defect sites and lead to integrated healing with fibrocartilaginous tissue by 6 weeks. Interestingly, fibrin with CTGF or fibrin with TGFβ3 resulted in a poor integration or a remained gap between the incised meniscus tissues. This is presently thought to indicate that a profibrogenic cue or a chondrogenic cue, alone, is insufficient to induce integrated healing of avascular meniscus tears and the sequentially controlled formation of intermediate fibrous integration followed by cartilaginous matrix remodeling is pivotal to guide integrative fibrocartilaginous meniscus healing. In addition, fibrin glue loaded with CTGF and TGFβ3-μS can be applied via a minimally invasive single injection. Together, the strategy to induce seamless healing of inner meniscus tears by temporal control of stem cell recruitment and differentiation may serve as an efficient and translational approach to overcome the current limitations of meniscus repair.

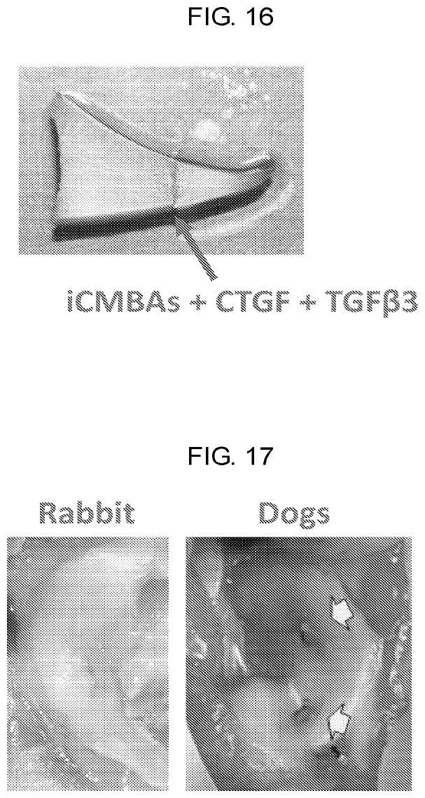

Further experiments were performed with a meniscus using injectable mussel-inspired biodegradable adhesives (iCMBA) with CTGF and TGFβ3 (see e.g., FIG. 16).

The above example describes a novel strategy to induce seamless healing of avascular inner meniscus tears by stem cell recruitment. As such, the described strategy to induce inner meniscus healing can overcome limitations of the current treatments of meniscus injuries.

1. A method of treating a subject having a tissue defect, comprising:
(a) providing a scaffold comprising (i) a matrix material, (ii) a chemotactic factor or a profibrogenic factor having a first release duration; and (iii) a chondrogenic factor having a second release duration longer than the first release duration;
(b) delivering the scaffold to a tissue defect site;
(c) forming a fibrous matrix at the tissue defect site by 6-14 days after scaffold delivery; and
(d) forming a fibrocartilaginous matrix comprising fibrochondrocyte cells or fibrochondrocyte-like cells integrated with tissue at the tissue defect site by 4-10 weeks after scaffold delivery.

2. The method of claim 1, wherein providing the scaffold comprises:
contacting the scaffold with the chemotactic factor or profibrogenic factor before delivering the scaffold to the tissue defect site; and
contacting the scaffold with the chondrogenic factor before delivering the scaffold to the tissue defect site.

3. The method of claim 1, wherein providing the scaffold comprises:
contacting the scaffold with the chemotactic factor or profibrogenic factor after delivering the scaffold to the tissue defect site; and
contacting the scaffold with the chondrogenic factor after delivering the scaffold to the tissue defect site and during or after the release duration of the chemotactic factor or profibrogenic factor.

4. The method of claim 1, wherein providing the scaffold comprises:
contacting the scaffold with the chemotactic factor or profibrogenic factor before delivering the scaffold to the tissue defect site; and
contacting the scaffold with the chondrogenic factor after delivering the scaffold to the tissue defect site and during or after the release duration of the chemotactic factor or profibrogenic factor.

5. The method of claim 1, wherein delivering the scaffold comprises placing the scaffold in fluid communication with a progenitor cell at the tissue defect site.

6. The method of claim 1, wherein the chemotactic factor or a profibrogenic factor comprises SDF-1, FGF-2, bFGF, CTGF, or GDF-5.

7. The method of claim 1, wherein the chemotactic factor or a profibrogenic factor comprises CTGF.

8. The method of claim 1, wherein,
the chemotactic factor or profibrogenic factor comprises CTGF;
the matrix material comprises a fibrin glue or an injectable mussel-inspired biodegradable adhesive (iCMBA); and
the CTGF is mixed with the matrix material.

9. The method of claim 1, wherein the chondrogenic factor comprises TGF-β1, TGF-β3, IGF-1, and BMP-7.

10. The method of claim 1, wherein the chondrogenic factor comprises TGF-β3.

11. The method of claim 1, wherein
the chondrogenic factor comprises TGF-β3;
TGF-β3 is encapsulated in polymeric microspheres; and
the encapsulated TGF-β3 is mixed with the matrix material.

12. The method of claim 1, wherein the matrix material comprises fibrin, fibrinogen, collagen, fibronectin, or injectable mussel-inspired biodegradable adhesive (iCMBA).

13. The method of claim 1, wherein the first release duration of the chemotactic factor or profibrogenic factor is at least about 4 days up to about 10 days.

14. The method of claim 13, wherein the second release duration of the chondrogenic factor is at least about 4 weeks.

15. The method of claim 1, wherein the second release duration of the chondrogenic factor is at least about 4 weeks.

16. The method of claim 1, wherein the scaffold:
(i) comprises an endogenous progenitor cell;
(ii) comprises an exogenous progenitor cell;
(iii) does not comprise an exogenous progenitor cell;
(iv) comprises a progenitor cell prior to scaffold delivery to the tissue defect site;
(v) does not comprise a progenitor cell until after scaffold delivery to the tissue defect site;
(vi) comprises an endogenous progenitor cell introduced to the scaffold in vivo or ex vivo; or
(vii) comprises an exogenous progenitor cell introduced to the scaffold in vivo or ex vivo.

17. The method of claim 1, wherein the tissue defect site is at least partially located in an inner or avascular region of a cartilaginous tissue.

18. The method of claim 1, wherein the tissue defect comprises a tear, injury, osteoarthritis, or degeneration.

19. The method of claim 1, wherein the tissue defect comprises a longitudinal or vertical tear, a radial tear, a horizontal tear, a bucket handle tear, a parrot beak tear, or a flap tear.

20. The method of claim 1, wherein the tissue is selected from the group consisting of a cartilaginous tissue, cartilage, a meniscus, a knee meniscus, a ligament, a ligament enthesis, a tendon, a tendon enthesis, an intervertebral disc, a temporomandibular joint (TMJ), a TMJ ligament, and a triangular fibrocartilage.

21. The method of claim 1, wherein the chemotactic factor or the profibrogenic factor is applied at a concentration of about 1 to about 1000 ng/mL.

22. The method of claim 1, wherein the chondrogenic factor is applied at a concentration of about 1 to about 1000 ng/mL.

23. A fibrocartilage tissue construct comprising:
(i) a progenitor cell;
(ii) an effective amount of a chemotactic factor or profibrogenic factor having a first release duration;
(iii) an effective amount of a chondrogenic factor having a second release duration; and
(iv) a scaffold comprising a matrix material,
wherein,
the first release duration is shorter than the second release duration;
the effective amount of the chemotactic factor or the profibrogenic factor induces migration of a progenitor cell into or onto the scaffold when the scaffold is in fluid communication with the progenitor cell; and
the effective amount of the chondrogenic factor in combination with the chemotactic factor or the profibrogenic factor induces formation of a fibrochondrocyte cell or a fibrochondrocyte-like cell from the progenitor cell.

24. A method of forming the fibrocartilage tissue construct of claim 23, comprising:
(i) providing a scaffold comprising a matrix material;
(ii) contacting the scaffold with a chemotactic factor or a profibrogenic factor;
(iii) contacting the scaffold comprising the chemotactic factor or the profibrogenic factor with a chondrogenic factor;
(iv) placing the scaffold in fluid communication with a progenitor cell; and
(v) delivering the scaffold to a tissue defect site;
wherein the combination of the chemotactic factor or profibrogenic factor and the chondrogenic factor induces formation of a fibrochondrocyte cell or a fibrochondrocyte-like cell at the tissue defect site.
